(19) United States
(12) Patent Application Publication
(10) Pub. No.: US 2023/0165571 A1
(43) Pub. Date: Jun. 1, 2023

(54) SAMPLE COLLECTION SWAB

(71) Applicant: PRESIDENT AND FELLOWS OF HARVARD COLLEGE, Cambridge, MA (US)

(72) Inventors: Richard NOVAK, Cambridge, MA (US); Donald E. INGBER, Cambridge, MA (US); Manuel Ramsés MARTÍNEZ FLORES, Cambridge, MA (US)

(73) Assignee: PRESIDENT AND FELLOWS OF HARVARD COLLEGE, Cambridge, MA (US)

(21) Appl. No.: 17/922,518

(22) PCT Filed: Mar. 19, 2021
(86) PCT No.: PCT/US2021/023107

§ 371 Date: Oct. 31, 2022

Related U.S. Application Data
(60) Provisional application No. 63/045,384, filed on Jun. 29, 2020.
(60) Provisional application No. 63/019,620, filed on May 4, 2020.
(60) Provisional application No. 63/018,685, filed on May 1, 2020.

(51) Int. Cl. A61B 10/00; A61F 13/38

(52) U.S. Cl. CPC A61B 10/0051; A61F 13/38

(57) ABSTRACT

The technology described herein is directed to a swab for sample collection. In one aspect, the swab comprises a sample collection head, which comprises a plurality of spaced annular rings. In one embodiment, the swab further comprises a tapered neck and a handle. In one embodiment, the swab is injection-molded using polypropylene. In other aspects, described herein are swabs comprising a water-soluble or biodegradable material. In additional aspects, described herein are kits comprising said swabs and methods of using said swabs.

### CROSS-REFERENCE TO RELATED APPLICATIONS

This application claims benefit under 35 U.S.C. § 119(e) of U.S. Provisional Application No. 63/018,685 filed May 1, 2020, U.S. Provisional Application No. 63/019,620 filed May 4, 2020, and U.S. Provisional Application No. 63/045,384 filed Jun. 29, 2020, the contents of each of which are incorporated herein by reference in their entireties.

### TECHNICAL FIELD

The technology described herein relates to a swab for sample collection.

### BACKGROUND

Nasopharyngeal (NP) swabs are used to detect respiratory infections, including but not limited to SARS-CoV-2. Swabs can also be used to collect samples from non-nasopharyngeal surfaces, such as the oropharyngeal, anterior nares, mid-turbinates, or buccal epithelial surface of a subject. There is a great need for swabs that exhibit at least the following attributes: (1) It is sufficiently rigid for collection of cells from the back of the throat. (2) It is sufficiently flexible for safety of use. (3) It collects adequate sample from the patient for subsequent tests (e.g., for viral infection). (4) It withstands the rigors of sterilization/disinfection without a) structural weakening, or b) chemically interfering with PCR testing. (5) It is compatible with standard PCR testing. Furthermore, there is great need for swabs that can be mass-produced inexpensively to address swab shortages.

### SUMMARY

The technology described herein is directed to a swab for sample collection. In one aspect, the swab comprises a sample collection head, which comprises a plurality of spaced annular rings. In one embodiment, the swab further comprises a tapered neck and a handle. In one embodiment, the swab is injection-molded using polypropylene. In other aspects, described herein are a swabs comprising a water-soluble or biodegradable material. Such a swab exhibits at least the following attributes: (1) It is sufficiently rigid for collection of cells from the back of the throat. (2) It is sufficiently flexible for safety of use. (3) It collects adequate sample from the patient for subsequent tests (e.g., for viral infection). (4) It withstands the rigors of sterilization/disinfection without a) structural weakening, or b) chemically interfering with PCR testing. (5) It is compatible with standard PCR testing. Furthermore, the swab can be mass-produced inexpensively to address swab shortages. In some embodiments, the swab or a portion of the swab is biodegradable and/or water-soluble, and does not interfere with downstream applications. Described herein are nasopharyngeal swabs, as well as non-nasopharyngeal swabs (e.g., anterior nare swabs). In additional aspects, described herein are kits comprising said swabs and methods of using said swabs.

In one aspect described herein is a swab comprising a sample collection head, the head comprising a plurality of spaced apart annular rings.

In some embodiments of any of the aspects, the plurality of rings are spaced 0.5 mm-2.0 mm.

In some embodiments of any of the aspects, the plurality of rings are spaced 0.75 mm.

In some embodiments of any of the aspects, the plurality of rings have a thickness of 0.1 mm-3.0 mm.

In some embodiments of any of the aspects, the plurality of rings have a thickness of 1.0 mm.

In some embodiments of any of the aspects, the plurality of rings have a diameter of 1.0 mm-4.0 mm.

In some embodiments of any of the aspects, the plurality of rings has a diameter of 2.5 mm.

In some embodiments of any of the aspects, the plurality of rings are tapered.

In some embodiments of any of the aspects, the plurality or rings have rounded edges.

In some embodiments of any of the aspects, the swab further comprises a handle and a neck, and wherein the head, handle, and neck comprise the same material.

In some embodiments of any of the aspects, at least one component of the swab is made from a different material from the remainder of the swab.

In some embodiments of any of the aspects, the swab is injection molded.

In some embodiments of any of the aspects, the material is a flexible polymer.

In some embodiments of any of the aspects, the material is polypropylene.

In some embodiments of any of the aspects, the material is biodegradable.

In some embodiments of any of the aspects, the material is water-soluble.

In some embodiments of any of the aspects, the material is polyvinyl alcohol (PVA).

In some embodiments of any of the aspects, the material is foam or a porous material.

In some embodiments of any of the aspects, the head does not comprise a fibrous coating.

In some embodiments of any of the aspects, the sample collection head comprises a first material, and the remainder of the swab comprises a second material.

In some embodiments of any of the aspects, the sample collection head comprises a water-soluble or biodegradable material and the remainder of the swab comprises a flexible polymer.

In some embodiments of any of the aspects, the sample collection head comprises PVA and the remainder of the swab comprises polypropylene.

In some embodiments of any of the aspects, the swab further comprises a neck, a plunger, and/or a flattened handle.

In some embodiments of any of the aspects, the neck tapers from a maximum diameter towards the handle to a minimum diameter towards the head.

In some embodiments of any of the aspects, the swab further comprises a breakpoint proximal to the head.

In some embodiments of any of the aspects, the head is stippled, roughened, or textured to increase surface area.

In one aspect described herein is a swab for sample collection, wherein the swab is constructed from a water-soluble or biodegradable material.

In some embodiments of any of the aspects, the material is biodegradable and water-soluble.

In some embodiments of any of the aspects, the material is polyvinyl alcohol (PVA).

In some embodiments of any of the aspects, the material is foam or a porous material.

In one aspect described herein is a swab for sample collection, wherein the swab is constructed from a flexible polymer and a water-soluble or biodegradable material.

In some embodiments of any of the aspects, the sample collection head comprises a water-soluble or biodegradable material and the remainder of the swab comprises a flexible polymer.

In some embodiments of any of the aspects, the sample collection head comprises PVA and the remainder of the swab comprises polypropylene.

In some embodiments of any of the aspects, the swab is in combination with a container tube.

In one aspect described herein is a kit comprising the swab of any one of claims 1-33.

In some embodiments of any of the aspects, the swab further comprises a container tube and/or sample transport media.

In one aspect described herein is a method of collecting a sample comprising contacting a sample with a swab as described herein.

In some embodiments of any of the aspects, the sample is selected from: nasopharyngeal, oropharyngeal, anterior nares, mid-turbinates, and buccal epithelial surface of a subject.

In some embodiments of any of the aspects, the sample is a nasopharyngeal epithelial surface of a subject.

In some embodiments of any of the aspects, the subject is infected with or suspected to be infected with a respiratory infection.

In some embodiments of any of the aspects, after the contacting step, the swab is separated into two pieces at the breakpoint.

In some embodiments of any of the aspects, after the contacting step, the swab is deposited into a container tube.

In some embodiments of any of the aspects, the container tube contains sample transport media.

In some embodiments of any of the aspects, after the contacting step, the swab or at least a portion of the swab is dissolved in a buffer.

In some embodiments of any of the aspects, the dissolved swab represents at most 22% (w/v) of the buffer.

In some embodiments of any of the aspects, the dissolved swab does not inhibit or reduce a downstream application.

### BRIEF DESCRIPTION OF THE DRAWINGS

FIG. 1 shows a mechanical drawing of an example nasopharyngeal (NP) swab (dimensions in mm), according to aspects of the present disclosure.

FIG. 2A shows a Computer Aided Design (CAD) drawing of the example NP swab of FIG. 1, according to aspects of the present disclosure.

FIG. 2B shows a zoomed-in view of the example swab’s sample collection head, according to aspects of the present disclosure. The arrow indicates an incomplete ring.

FIG. 3A shows an image of injection-molded samples of the example NP swab, according to aspects of the present disclosure.

FIG. 3B shows a demonstration of the flexibility of injection-molded versions of the NP swab, according to aspects of the present disclosure.

FIG. 4 shows use of flocked and unflocked swabs to test for release of glyceraldehyde 3-phosphate dehydrogenase (GAPDH) from nasal samples from volunteers, according to aspects of the present disclosure. The example NP swab described herein is #3 (see e.g., Table 4). The other numbers are other swabs that have been approved for use. Note that all swabs tested performed about the same in amount of GAPDH detected.

FIG. 5 shows a schematic of an example non-nasopharyngeal (e.g., anterior nares) swab comprising an integrated plunger as a stopper to use for dry transport, according to aspects of the present disclosure.

FIG. 6 shows an example of geometry for an example swab comprising an integrated plunger (dimensions in mm), according to aspects of the present disclosure.

FIG. 7 is a bar graph showing PVA RT-qPCR compatibility, according to aspects of the present disclosure. The x-axis indicates the final PVA percent concentration (w/v%) in the reaction. Bars within each PVA percentage group are in the same order left-right as the order of the legend left-right_{.}

FIG. 8 is a line graph showing PVA RPA-qPCR compatibility, according to aspects of the present disclosure.

FIG. 9 is a bar graph showing PVA RT-qPCR compatibility using the QIAmp™ Viral RNA Mini Kit for RNA purification, according to aspects of the present disclosure.

### DETAILED DESCRIPTION

The technology described herein is directed to a swab for sample collection. In one aspect, the swab comprises a sample collection head, which comprises a plurality of spaced annular rings. In one embodiment, the swab further comprises a tapered neck and a handle. In one embodiment, the swab is injection-molded using polypropylene. In other aspects, described herein are a swabs comprising a water-soluble or biodegradable material. Such a swab exhibits at least the following attributes: (1) It is sufficiently rigid for collection of cells from the back of the throat. (2) It is sufficiently flexible for safety of use. (3) It collects adequate sample from the patient for subsequent tests (e.g., for viral infection). (4) It withstands the rigors of sterilization/disinfection without a) structural weakening, or b) chemically interfering with PCR testing. (5) It is compatible with standard PCR testing. Furthermore, the swab can be mass-produced inexpensively to address swab shortages. In some embodiments, the swab or a portion of the swab is biodegradable and/or water-soluble, and does not interfere with downstream applications. Described herein are nasopharyngeal swabs, as well as non-nasopharyngeal swabs (e.g., anterior nare swabs). In additional aspects, described herein are kits comprising said swabs and methods of using said swabs.

### Swab

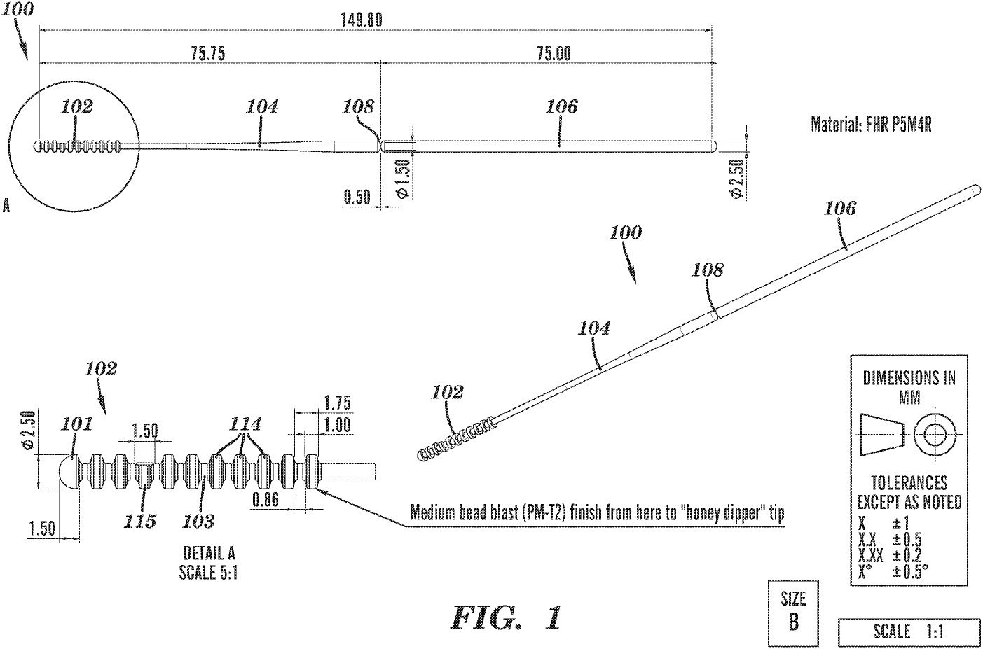

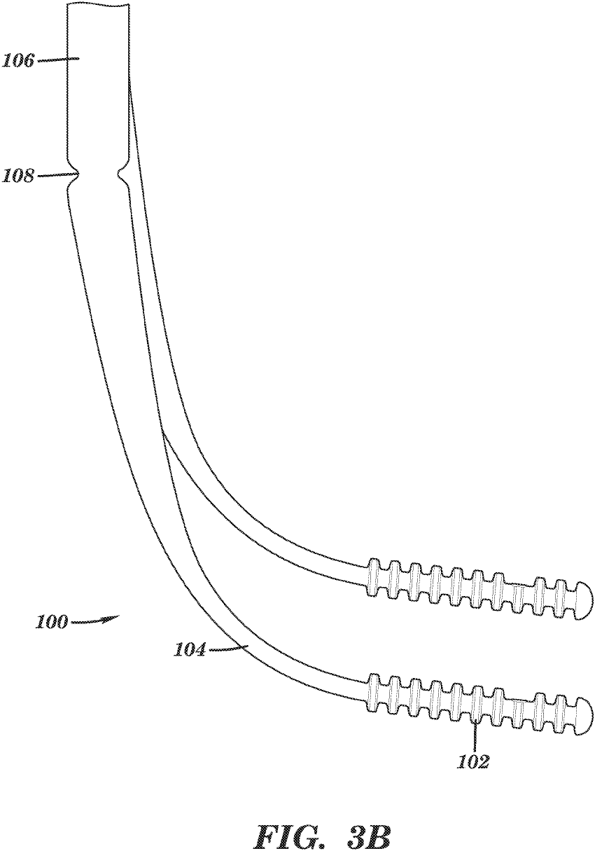

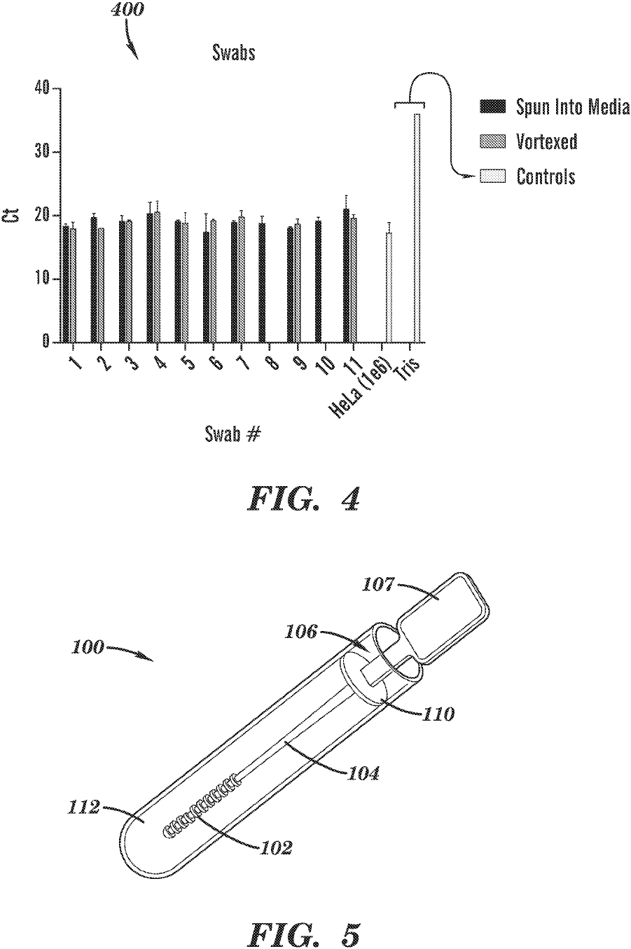

Described herein is a swab 100 for sample collection, as illustrated in FIG. 1. In one aspect, the swab 100 comprises a sample collection head 102. In some embodiments, the swab 100 further comprises a neck 104. In some embodiments, the swab 100 further comprises a handle 106. In some embodiments, the swab 100 further comprises a breakpoint 108. In some embodiments, the swab 100 further comprises a plunger 110 (FIGS. 5 and 6). In some embodiments, the swab 100 is in combination with a container tube 112 (FIG. 5). Any combination of the foregoing is contemplated herein. Exemplary combinations are shown in Table 3 below.

TABLE 3

Exemplary Swabs (an “X” indicates that the swab 100 comprises the indicated component; tube indicates the container tube 112 with which the swab 100 can be in combination)

| | | | | | |
|---|---|---|---|---|---|
| Head (102) | Neck (104) | Handle (106) | Breakpoint (108) | Plunger (110) | Tube (112) |
| X | | | | | |
| X | X | | | | |
| X | | X | | | |
| X | X | X | | | |
| X | | | X | | |
| X | X | | X | | |
| X | | X | X | | |
| X | X | X | X | | |
| X | | | | X | |
| X | X | | | X | |
| X | | X | | X | |
| X | X | X | | X | |
| X | | | X | X | |
| X | X | | X | X | |
| X | | X | X | X | |
| X | X | X | X | X | |
| X | | | | | X |
| X | X | | | | X |
| X | | X | | | X |
| X | X | X | | | X |
| X | | | X | | X |
| X | X | | X | | X |
| X | | X | X | | X |
| X | X | X | X | | X |
| X | | | | X | X |
| X | X | | | X | X |
| X | | X | | X | X |
| X | X | X | | X | X |
| X | | | X | X | X |
| X | X | | X | X | X |
| X | | X | X | X | X |
| X | X | X | X | X | X |

The components of the swab 100 can be in any order. In some embodiments of any of the aspects, the swab 100 comprises in the following order: head-neck-handle, with optional components inserted into this order. Non limiting examples of ordered components of the swab 100 include: head-neck-handle; head-neck-breakpoint-handle; head-breakpoint-neck-handle; head-neck-breakpoint-neck-handle; head-neck-plunger-handle; head-plunger-neck-handle; head-neck-plunger-neck-handle; head-neck-breakpoint-plunger-handle; head-neck-plunger-breakpoint-handle; head-neck-breakpoint-neck-plunger-handle; and head-neck-plunger-neck-breakpoint-handle.

In some embodiments, the components of the swab 100 are directly or indirectly connected to each other. In some embodiments, the components of the swab 100 are aligned according to the same central axis (e.g., share the same cross-sectional midpoint).

In some embodiments, the length of the swab 100 (e.g., from “distal” end, which is used herein to refer to the head end, to the “proximal” end, which is used herein to refer to the non-head end, such as the handle end) is at least 140 mm. In some embodiments, the length of the swab 100 is about 150 mm (see e.g., FIG. 1). As a non-limiting example, the swab 100 is a nasopharyngeal swab of a sufficient length (e.g., about 150 mm) to reach the nasopharynx of the subject. In some embodiments, the length of the swab 100 is at least 70 mm. In some embodiments, the length of the swab 100 is about 75 mm (see e.g., FIG. 6). As a non-limiting example, the swab 100 is a non-nasopharyngeal swab of a sufficient length (e.g., about 75 mm) to reach the non-nasopharyngeal surface of the subject, e.g., oropharyngeal, anterior nares, mid-turbinates, or buccal epithelial surface of a subject. In some embodiments, the length of the swab 100 is at least 50 mm, at least 55 mm, at least 60 mm, at least 65 mm, at least 70 mm, at least 75 mm, at least 80 mm, at least 85 mm, at least 90 mm, at least 95 mm, at least 100 mm, at least 105 mm, at least 110 mm, at least 115 mm, at least 120 mm, at least 125 mm, at least 130 mm, at least 135 mm, at least 140 mm, at least 145 mm, at least 150 mm, at least 155 mm, at least 160 mm, at least 165 mm, at least 170 mm, at least 175 mm, at least 180 mm, at least 185 mm, at least 190 mm, at least 195 mm, at least 200 mm, at least 205 mm, at least 210 mm, at least 215 mm, at least 220 mm, at least 225 mm, at least 230 mm, at least 235 mm, at least 240 mm, at least 245 mm, at least 250 mm, at least 255 mm, at least 260 mm, at least 265 mm, at least 270 mm, at least 275 mm, at least 280 mm, at least 285 mm, at least 290 mm, at least 295 mm, or at least 300 mm.

In some embodiments, the length of the swab 100 is at most 140 mm. In some embodiments, the length of the swab 100 is at most 50 mm, at most 55 mm, at most 60 mm, at most 65 mm, at most 70 mm, at most 75 mm, at most 80 mm, at most 85 mm, at most 90 mm, at most 95 mm, at most 100 mm, at most 105 mm, at most 110 mm, at most 115 mm, at most 120 mm, at most 125 mm, at most 130 mm, at most 135 mm, at most 140 mm, at most 145 mm, at most 150 mm, at most 155 mm, at most 160 mm, at most 165 mm, at most 170 mm, at most 175 mm, at most 180 mm, at most 185 mm, at most 190 mm, at most 195 mm, at most 200 mm, at most 205 mm, at most 210 mm, at most 215 mm, at most 220 mm, at most 225 mm, at most 230 mm, at most 235 mm, at most 240 mm, at most 245 mm, at most 250 mm, at most 255 mm, at most 260 mm, at most 265 mm, at most 270 mm, at most 275 mm, at most 280 mm, at most 285 mm, at most 290 mm, at most 295 mm, or at most 300 mm.

In some embodiments, the swab 100 is in combination with a container tube 112 (see e.g., FIG. 5). In some embodiments, the swab 100 is inserted into the container tube 112. In some embodiments, the container tube 112 contains sample transport media. In some embodiments, the container tube 112 can be constructed from a transparent material. In some embodiments, the container tube 112 has a length that is the same as the total length of the swab 100. In some embodiments, the container tube 112 has a length that is less than the total length of the swab 100. In some embodiments, the container tube 112 has a length that is greater than the total length of the swab 100. In some embodiments, the container tube 112 has an internal diameter that is greater than the maximum diameter of the swab 100. In some embodiments, the container tube 112 has an internal diameter that is the same as the maximum diameter of the swab 100 (e.g., the maximum diameter of the plunger 110). In some embodiments, the container tube 112 comprises internal flanges. In some embodiments, the container tube 112 comprises internal grooves. In some embodiments, the container tube 112 comprises an internal geometric feature to permit snapping, holding in place, and/or sealing the swab 100 and biological sample within the container tube 112.

In some embodiments, the swab 100 comprises a barcode or label. In some embodiments, the barcode or label can be located on any component of the swab 100, e.g., the sample collection head 102, the neck 104, the handle 106, the plunger 110, or the container tube 112. In some embodiments, the barcode or label is located on a flattened portion 107 of the handle 106. In some embodiments, the barcode or label is unique to each sample and permits identification of the sample.

### Sample Collection Head

In one aspect, the swab 100 comprises a sample collection head 102. As used herein, the term “sample collection head” (or simply “head”) refers to the distal end of the swab 100, e.g., that is contacted with a sample to be collected; as described herein, at least a portion of the sample (e.g., mucus, cells, and microorganisms) is collected in the head 102 of the swab 100, which can be used for downstream application.

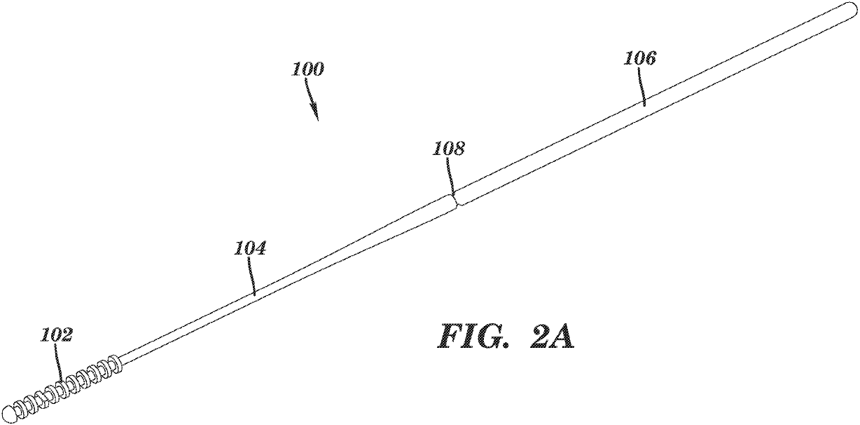

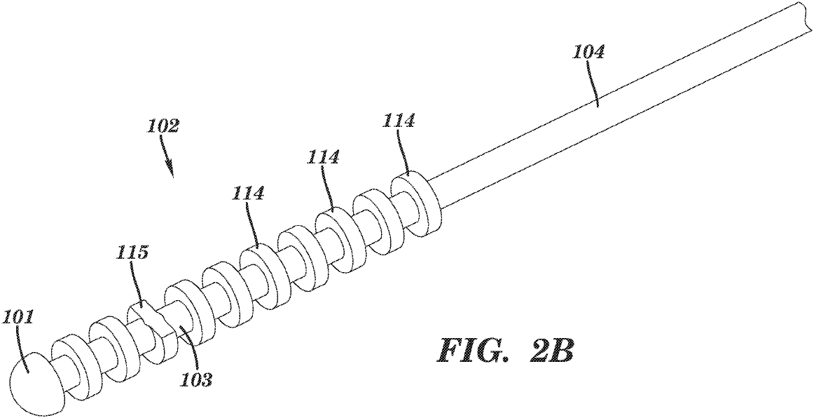

In some embodiments, the sample collection head 102 comprises a plurality of spaced annular rings 114 (see e.g., FIG. 1 and FIGS. 2A-2B). As used herein, the term “annular ring” or “ring” refers to a projection that has a greater diameter than the diameter of an axial shaft 103 of the collection head 102. As used herein, the term “axial shaft” refers to sections that connect or “run through” the spaced rings 114; the axial shaft 103 can be continuous with the neck 104 and/or handle 106 of the swab 100. In some embodiments, the plurality of rings 114 comprises 2, 3, 4, 5, 6, 7, 8, 9, 10, 11, 12, 13, 14, 15, 16, 17, 18, 19, 20, 21, 22, 23, 24, 25, or more rings 114. In some embodiments, the plurality of rings 114 comprises 10 rings 114.

In some embodiments, the cross-section of the rings 114 is a circle, a semicircle, a truncated circle, or a circle with one or more flat sides. In some embodiments, the cross-section of the rings 114 is circular. In some embodiments, the rings 114 have a polygonal cross section, e.g., a cross-section in the shape of a triangle, a square, a quadrilateral, a trapezoid, a pentagon, a hexagon, or a polygon with at least 7, 8, 9, 10, 11, 12, 13, 14, 15, 16, 17, 18, 19, 20 or more sides. In some embodiments, at least one side of the cross-section of the rings 114 comprises a convex and/or concave curve. In some embodiments, the cross section of the rings 114 is a rotationally symmetric shape. In some embodiments, the cross section of the rings 114 is an asymmetric shape. In some embodiments, the cross-section of each of the plurality of rings 114 is the same every other one of the plurality of rings 114. In some embodiments, the cross-section of at least one of the plurality of rings 114 is different for at least one ring 114 in the plurality of rings 114; the head 102 can comprise any combination of different (e.g., at least 2, at least 3, at least 4, at least 5) ring cross-sections.

In some embodiments, the cross-section of the axial shaft 103 is a circle, a semicircle, a truncated circle, or a circle with one or more flat sides. In some embodiments, the cross-section of the axial shaft 103 is circular. In some embodiments, the axial shaft 103 comprises a cylindrical rod. In some embodiments, the axial shaft 103 has a polygonal cross section, e.g., a cross-section in the shape of a triangle, a square, a quadrilateral, a trapezoid, a pentagon, a hexagon, or a polygon with at least 7, 8, 9, 10, 11, 12, 13, 14, 15, 16, 17, 18, 19, 20 or more sides. In some embodiments, at least one side of the cross-section of the axial shaft 103 comprises a convex and/or concave curve. In some embodiments, the cross section of the axial shaft 103 is a rotationally symmetric shape. In some embodiments, the cross section of the axial shaft 103 is an asymmetric shape. In some embodiments, the cross-section of the axial shaft 103 is the same for the entirety of the axial shaft 103. In some embodiments, the cross-section of the axial shaft 103 is different for at least one portion of the axial shaft 103; the axial shaft 103 can comprise any combination of different (e.g., at least 2, at least 3, at least 4, at least 5) axial shaft cross-sections.

In some embodiments, the plurality of rings 114 is spaced apart, e.g., exposing the axial shaft 103. As used herein, ring spacing refers to the distance between the end of one of the rings 114 to the beginning of the next one of the rings 114. In some embodiments, the plurality of rings 114 is spaced 0.1 mm-3.0 mm. In some embodiments, the plurality of rings 114 is spaced 0.5 mm-2.0 mm. In some embodiments, the plurality of rings 114 is spaced 0.75 mm. In some embodiments, the plurality of rings 114 is spaced at least 0.1 mm, at least 0.15 mm, at least 0.2 mm, at least 0.25 mm, at least 0.3 mm, at least 0.35 mm, at least 0.4 mm, at least 0.45 mm, at least 0.5 mm, at least 0.55 mm, at least 0.6 mm, at least 0.65 mm, at least 0.7 mm, at least 0.75 mm, at least 0.8 mm, at least 0.85 mm, at least 0.9 mm, at least 0.95 mm, at least 1 mm, at least 1.05 mm, at least 1.1 mm, at least 1.15 mm, at least 1.2 mm, at least 1.25 mm, at least 1.3 mm, at least 1.35 mm, at least 1.4 mm, at least 1.45 mm, at least 1.5 mm, at least 1.55 mm, at least 1.6 mm, at least 1.65 mm, at least 1.7 mm, at least 1.75 mm, at least 1.8 mm, at least 1.85 mm, at least 1.9 mm, at least 1.95 mm, at least 2 mm, at least 2.05 mm, at least 2.1 mm, at least 2.15 mm, at least 2.2 mm, at least 2.25 mm, at least 2.3 mm, at least 2.35 mm, at least 2.4 mm, at least 2.45 mm, at least 2.5 mm, at least 2.55 mm, at least 2.6 mm, at least 2.65 mm, at least 2.7 mm, at least 2.75 mm, at least 2.8 mm, at least 2.85 mm, at least 2.9 mm, at least 2.95 mm, or at least 3 mm. In some embodiments, the spacing between each sequential pair of rings of the plurality of rings 114 is the same for all pairs in the head 102. In some embodiments, the spacing between each sequential pair of rings of the plurality of rings 114 is different for at least one of the pairs in the head 102; the head 102 can comprise any combination of different (e.g., at least 2, at least 3, at least 4, at least 5) ring spacing distances.

In some embodiments, the plurality of rings 114 have a thickness of 0.1 mm-3.0 mm. As used herein, ring thickness refers to the distance from the beginning of one ring of the plurality of rings 114 to the end of that same ring. In some embodiments, the plurality of rings 114 have a thickness of 1.0 mm. In some embodiments, the plurality of rings 114 have a thickness of 0.5 mm-2.0 mm. In some embodiments, the plurality of rings 114 have a thickness of 0.75 mm. In some embodiments, the plurality of rings 114 have a thickness of at least 0.1 mm, at least 0.15 mm, at least 0.2 mm, at least 0.25 mm, at least 0.3 mm, at least 0.35 mm, at least 0.4 mm, at least 0.45 mm, at least 0.5 mm, at least 0.55 mm, at least 0.6 mm, at least 0.65 mm, at least 0.7 mm, at least 0.75 mm, at least 0.8 mm, at least 0.85 mm, at least 0.9 mm, at least 0.95 mm, at least 1 mm, at least 1.05 mm, at least 1.1 mm, at least 1.15 mm, at least 1.2 mm, at least 1.25 mm, at least 1.3 mm, at least 1.35 mm, at least 1.4 mm, at least 1.45 mm, at least 1.5 mm, at least 1.55 mm, at least 1.6 mm, at least 1.65 mm, at least 1.7 mm, at least 1.75 mm, at least 1.8 mm, at least 1.85 mm, at least 1.9 mm, at least 1.95 mm, at least 2 mm, at least 2.05 mm, at least 2.1 mm, at least 2.15 mm, at least 2.2 mm, at least 2.25 mm, at least 2.3 mm, at least 2.35 mm, at least 2.4 mm, at least 2.45 mm, at least 2.5 mm, at least 2.55 mm, at least 2.6 mm, at least 2.65 mm, at least 2.7 mm, at least 2.75 mm, at least 2.8 mm, at least 2.85 mm, at least 2.9 mm, at least 2.95 mm, or at least 3 mm. In some embodiments, the ring thickness is the same for each of the plurality of rings 114. In some embodiments, at least one ring of the plurality of rings 114 is a different thickness than another ring in the plurality of rings 114; the head 102 can comprise any combination of different (e.g., at least 2, at least 3, at least 4, at least 5) ring thicknesses.

In some embodiments, the plurality of rings 114 have a thickness of at most 0.1 mm, at most 0.15 mm, at most 0.2 mm, at most 0.25 mm, at most 0.3 mm, at most 0.35 mm, at most 0.4 mm, at most 0.45 mm, at most 0.5 mm, at most 0.55 mm, at most 0.6 mm, at most 0.65 mm, at most 0.7 mm, at most 0.75 mm, at most 0.8 mm, at most 0.85 mm, at most 0.9 mm, at most 0.95 mm, at most 1 mm, at most 1.05 mm, at most 1.1 mm, at most 1.15 mm, at most 1.2 mm, at most 1.25 mm, at most 1.3 mm, at most 1.35 mm, at most 1.4 mm, at most 1.45 mm, at most 1.5 mm, at most 1.55 mm, at most 1.6 mm, at most 1.65 mm, at most 1.7 mm, at most 1.75 mm, at most 1.8 mm, at most 1.85 mm, at most 1.9 mm, at most 1.95 mm, at most 2 mm, at most 2.05 mm, at most 2.1 mm, at most 2.15 mm, at most 2.2 mm, at most 2.25 mm, at most 2.3 mm, at most 2.35 mm, at most 2.4 mm, at most 2.45 mm, at most 2.5 mm, at most 2.55 mm, at most 2.6 mm, at most 2.65 mm, at most 2.7 mm, at most 2.75 mm, at most 2.8 mm, at most 2.85 mm, at most 2.9 mm, at most 2.95 mm, or at most 3 mm.

In some embodiments, the plurality of rings 114 have a diameter of 1.0 mm-4.0 mm. As used herein, the term “diameter” refers to the distance of a straight line passing through the axial center of a circular cross section (e.g., taken perpendicular to the axial shaft 103). In some embodiments, the plurality of rings 114 have a diameter of 2.5 mm. In some embodiments, the plurality of rings 114 have a diameter of 1.0 mm. In some embodiments, the plurality of rings 114 have a diameter of at least 1 mm, at least 1.05 mm, at least 1.1 mm, at least 1.15 mm, at least 1.2 mm, at least 1.25 mm, at least 1.3 mm, at least 1.35 mm, at least 1.4 mm, at least 1.45 mm, at least 1.5 mm, at least 1.55 mm, at least 1.6 mm, at least 1.65 mm, at least 1.7 mm, at least 1.75 mm, at least 1.8 mm, at least 1.85 mm, at least 1.9 mm, at least 1.95 mm, at least 2 mm, at least 2.05 mm, at least 2.1 mm, at least 2.15 mm, at least 2.2 mm, at least 2.25 mm, at least 2.3 mm, at least 2.35 mm, at least 2.4 mm, at least 2.45 mm, at least 2.5 mm, at least 2.55 mm, at least 2.6 mm, at least 2.65 mm, at least 2.7 mm, at least 2.75 mm, at least 2.8 mm, at least 2.85 mm, at least 2.9 mm, at least 2.95 mm, at least 3 mm, at least 3.05 mm, at least 3.1 mm, at least 3.15 mm, at least 3.2 mm, at least 3.25 mm, at least 3.3 mm, at least 3.35 mm, at least 3.4 mm, at least 3.45 mm, at least 3.5 mm, at least 3.55 mm, at least 3.6 mm, at least 3.65 mm, at least 3.7 mm, at least 3.75 mm, at least 3.8 mm, at least 3.85 mm, at least 3.9 mm, at least 3.95 mm, or at least 4.0 mm. In some embodiments, the ring diameter is the same for each ring of the plurality of rings 114. In some embodiments, at least one ring is a different diameter than another ring in the plurality of rings 114; the head 102 can comprise any combination of different (e.g., at least 2, at least 3, at least 4, at least 5) ring diameters.

In some embodiments, the plurality of rings 114 have a diameter that is less than the narrowest section of the nasal cavity (e.g., less than 4 mm). In some embodiments, the plurality of rings 114 have a diameter of at most 1 mm, at most 1.05 mm, at most 1.1 mm, at most 1.15 mm, at most 1.2 mm, at most 1.25 mm, at most 1.3 mm, at most 1.35 mm, at most 1.4 mm, at most 1.45 mm, at most 1.5 mm, at most 1.55 mm, at most 1.6 mm, at most 1.65 mm, at most 1.7 mm, at most 1.75 mm, at most 1.8 mm, at most 1.85 mm, at most 1.9 mm, at most 1.95 mm, at most 2 mm, at most 2.05 mm, at most 2.1 mm, at most 2.15 mm, at most 2.2 mm, at most 2.25 mm, at most 2.3 mm, at most 2.35 mm, at most 2.4 mm, at most 2.45 mm, at most 2.5 mm, at most 2.55 mm, at most 2.6 mm, at most 2.65 mm, at most 2.7 mm, at most 2.75 mm, at most 2.8 mm, at most 2.85 mm, at most 2.9 mm, at most 2.95 mm, at most 3 mm, at most 3.05 mm, at most 3.1 mm, at most 3.15 mm, at most 3.2 mm, at most 3.25 mm, at most 3.3 mm, at most 3.35 mm, at most 3.4 mm, at most 3.45 mm, at most 3.5 mm, at most 3.55 mm, at most 3.6 mm, at most 3.65 mm, at most 3.7 mm, at most 3.75 mm, at most 3.8 mm, at most 3.85 mm, at most 3.9 mm, at most 3.95 mm, or at most 4 mm.

In some embodiments, the axial shaft 103 has a diameter of 0.5 mm-4.0 mm. By definition, the diameter of the axial shaft 103 is less than the diameter of the plurality of rings 114. In some embodiments, the axial shaft 103 has a diameter of 1.2 mm. In some embodiments, the axial shaft 103 has a diameter of at least 0.5 mm, at least 0.55 mm, at least 0.6 mm, at least 0.65 mm, at least 0.7 mm, at least 0.75 mm, at least 0.8 mm, at least 0.85 mm, at least 0.9 mm, at least 0.95 mm, at least 1 mm, at least 1.05 mm, at least 1.1 mm, at least 1.15 mm, at least 1.2 mm, at least 1.25 mm, at least 1.3 mm, at least 1.35 mm, at least 1.4 mm, at least 1.45 mm, at least 1.5 mm, at least 1.55 mm, at least 1.6 mm, at least 1.65 mm, at least 1.7 mm, at least 1.75 mm, at least 1.8 mm, at least 1.85 mm, at least 1.9 mm, at least 1.95 mm, at least 2 mm, at least 2.05 mm, at least 2.1 mm, at least 2.15 mm, at least 2.2 mm, at least 2.25 mm, at least 2.3 mm, at least 2.35 mm, at least 2.4 mm, at least 2.45 mm, at least 2.5 mm, at least 2.55 mm, at least 2.6 mm, at least 2.65 mm, at least 2.7 mm, at least 2.75 mm, at least 2.8 mm, at least 2.85 mm, at least 2.9 mm, at least 2.95 mm, at least 3 mm, at least 3.05 mm, at least 3.1 mm, at least 3.15 mm, at least 3.2 mm, at least 3.25 mm, at least 3.3 mm, at least 3.35 mm, at least 3.4 mm, at least 3.45 mm, at least 3.5 mm, at least 3.55 mm, at least 3.6 mm, at least 3.65 mm, at least 3.7 mm, at least 3.75 mm, at least 3.8 mm, at least 3.85 mm, at least 3.9 mm, at least 3.95 mm, or at least 4.0 mm. In some embodiments, the axial shaft 103 diameter is constant throughout the head 102. In some embodiments, the axial shaft diameter is the same diameter as the diameter of the distal region of the neck 104. In some embodiments, at least one portion of the axial shaft 103 is a different diameter than portion of the axial shaft 103; the axial shaft 103 can comprise any combination of different (e.g., at least 2, at least 3, at least 4, at least 5) diameters.

In some embodiments, the axial shaft 103 has a diameter of at most 0.5 mm, at most 0.55 mm, at most 0.6 mm, at most 0.65 mm, at most 0.7 mm, at most 0.75 mm, at most 0.8 mm, at most 0.85 mm, at most 0.9 mm, at most 0.95 mm, at most 1 mm, at most 1.05 mm, at most 1.1 mm, at most 1.15 mm, at most 1.2 mm, at most 1.25 mm, at most 1.3 mm, at most 1.35 mm, at most 1.4 mm, at most 1.45 mm, at most 1.5 mm, at most 1.55 mm, at most 1.6 mm, at most 1.65 mm, at most 1.7 mm, at most 1.75 mm, at most 1.8 mm, at most 1.85 mm, at most 1.9 mm, at most 1.95 mm, at most 2 mm, at most 2.05 mm, at most 2.1 mm, at most 2.15 mm, at most 2.2 mm, at most 2.25 mm, at most 2.3 mm, at most 2.35 mm, at most 2.4 mm, at most 2.45 mm, at most 2.5 mm, at most 2.55 mm, at most 2.6 mm, at most 2.65 mm, at most 2.7 mm, at most 2.75 mm, at most 2.8 mm, at most 2.85 mm, at most 2.9 mm, at most 2.95 mm, at most 3 mm, at most 3.05 mm, at most 3.1 mm, at most 3.15 mm, at most 3.2 mm, at most 3.25 mm, at most 3.3 mm, at most 3.35 mm, at most 3.4 mm, at most 3.45 mm, at most 3.5 mm, at most 3.55 mm, at most 3.6 mm, at most 3.65 mm, at most 3.7 mm, at most 3.75 mm, at most 3.8 mm, at most 3.85 mm, at most 3.9 mm, or at most 3.95 mm.

As described herein, the term “annular ring” or “ring” refers to a circular projection that has a greater diameter than the diameter of an axial shaft 103 of the collection head 102. Accordingly, the height of a ring 114 (e.g., from the axial shaft 103 to the widest diameter of the ring 114) can be calculated as half of the difference between the diameter of the ring 114 and the diameter of the axial shaft 103. In some embodiments, the plurality of rings 114 have a height of 0.5 mm-1.75 mm. In some embodiments, the plurality of rings 114 have a height of 0.65 mm (e.g., 0.5*(2.5-1.2)). In some embodiments, the plurality of rings 114 have a height of at least 0.5 mm, at least 0.51 mm, at least 0.52 mm, at least 0.53 mm, at least 0.54 mm, at least 0.55 mm, at least 0.56 mm, at least 0.57 mm, at least 0.58 mm, at least 0.59 mm, at least 0.6 mm, at least 0.61 mm, at least 0.62 mm, at least 0.63 mm, at least 0.64 mm, at least 0.65 mm, at least 0.66 mm, at least 0.67 mm, at least 0.68 mm, at least 0.69 mm, at least 0.7 mm, at least 0.71 mm, at least 0.72 mm, at least 0.73 mm, at least 0.74 mm, at least 0.75 mm, at least 0.76 mm, at least 0.77 mm, at least 0.78 mm, at least 0.79 mm, at least 0.8 mm, at least 0.81 mm, at least 0.82 mm, at least 0.83 mm, at least 0.84 mm, at least 0.85 mm, at least 0.86 mm, at least 0.87 mm, at least 0.88 mm, at least 0.89 mm, at least 0.9 mm, at least 0.91 mm, at least 0.92 mm, at least 0.93 mm, at least 0.94 mm, at least 0.95 mm, at least 0.96 mm, at least 0.97 mm, at least 0.98 mm, at least 0.99 mm, at least 1.0 mm, at least 1.05 mm, at least 1.1 mm, at least 1.15 mm, at least 1.2 mm, at least 1.25 mm, at least 1.3 mm, at least 1.35 mm, at least 1.4 mm, at least 1.45 mm, at least 1.5 mm, at least 1.55 mm, at least 1.6 mm, at least 1.65 mm, at least 1.7 mm, or at least 1.75 mm. In some embodiments, the ring height is the same for the plurality of rings 114. In some embodiments, at least one ring 114 is a different height than another ring 114 in the plurality of rings 114; the head 102 can comprise any combination of different (e.g., at least 2, at least 3, at least 4, at least 5) ring heights.

In some embodiments, the plurality of rings 114 have a height of at most 0.5 mm, at most 0.51 mm, at most 0.52 mm, at most 0.53 mm, at most 0.54 mm, at most 0.55 mm, at most 0.56 mm, at most 0.57 mm, at most 0.58 mm, at most 0.59 mm, at most 0.6 mm, at most 0.61 mm, at most 0.62 mm, at most 0.63 mm, at most 0.64 mm, at most 0.65 mm, at most 0.66 mm, at most 0.67 mm, at most 0.68 mm, at most 0.69 mm, at most 0.7 mm, at most 0.71 mm, at most 0.72 mm, at most 0.73 mm, at most 0.74 mm, at most 0.75 mm, at most 0.76 mm, at most 0.77 mm, at most 0.78 mm, at most 0.79 mm, at most 0.8 mm, at most 0.81 mm, at most 0.82 mm, at most 0.83 mm, at most 0.84 mm, at most 0.85 mm, at most 0.86 mm, at most 0.87 mm, at most 0.88 mm, at most 0.89 mm, at most 0.9 mm, at most 0.91 mm, at most 0.92 mm, at most 0.93 mm, at most 0.94 mm, at most 0.95 mm, at most 0.96 mm, at most 0.97 mm, at most 0.98 mm, at most 0.99 mm, at most 1.0 mm, at most 1.05 mm, at most 1.1 mm, at most 1.15 mm, at most 1.2 mm, at most 1.25 mm, at most 1.3 mm, at most 1.35 mm, at most 1.4 mm, at most 1.45 mm, at most 1.5 mm, at most 1.55 mm, at most 1.6 mm, at most 1.65 mm, at most 1.7 mm, or at most 1.75 mm.

In some embodiments, the plurality of rings 114, or at least a portion of the plurality of rings 114, are tapered, e.g., have sequentially reduced diameters towards one end, both ends, or from the middle of the plurality of rings 114. In some embodiments, the plurality of rings 114 taper from a maximum diameter at the distal end of the head 102 to a minimum diameter at the proximal end of the head 102 (e.g., closer to the neck 104 or handle 106). In some embodiments, the plurality of rings 114 taper from a minimum diameter at the distal end of the head 102 to a maximum diameter at the proximal end of the head 102. In some embodiments, the maximum diameter of the plurality of rings 114 occurs at a middle ring 114 or rings 114 of the head 102 and the diameters taper to a minimum diameter at the proximal and/or distal(s) end of the head 102. In some embodiments, the minimum diameter of the plurality of rings 114 occurs at a middle ring 114 or rings 114 of the head 102 and the diameters taper to a maximum diameter at the proximal and/or distal end(s) of the head 102. In some embodiments, the rings 114 alternate between a minimum diameter and a maximum diameter.

In some embodiments, the axial shaft 103, or at least a portion of the axial shaft 103, is tapered, e.g., has sequentially reduced diameters towards one end, both ends, or from the middle of the axial shaft 103. In some embodiments, the axial shaft 103 tapers from a maximum diameter at the distal end of the axial shaft 103 to a minimum diameter at the proximal end of the axial shaft 103 (e.g., closer to the neck 104 or handle 106). In some embodiments, the axial shaft 103 tapers from a minimum diameter at the distal end of the axial shaft 103 to a maximum diameter at the proximal end of the axial shaft 103. In some embodiments, the maximum diameter of the axial shaft 103 occurs in the middle of the head 102 and the diameters taper to a minimum diameter at the proximal and/or distal(s) end of the axial shaft 103. In some embodiments, the minimum diameter of the axial shaft 103 occurs in the middle of the head 102 and the diameters taper to a maximum diameter at the proximal and/or distal end(s) of the axial shaft 103. In some embodiments, the axial shaft 103 alternates between a minimum diameter and a maximum diameter.

In some embodiments, the plurality of rings 114 have rounded edges, e.g., have eased, curved, and/or non-angular edge. In some embodiments, the rounding of the rings 114 is manufactured using an abrasion method (e.g., bead blasting, sandpaper) and/or a mold (e.g., an injection mold). In some embodiments, the rounding of the rings 114 facilitates insertion and withdrawal into the sample or subject. In some embodiments, the distance between the rounded end edge of a first ring 114 to the rounded beginning edge of the next proximate second ring 114 is at least 0.75 mm. In some embodiments, the distance between the rounded end edge of a first ring 114 to the rounded beginning edge of the next proximate second ring 114 is 0.86 mm (see e.g., FIG. 1). In some embodiments, the distance between the rounded end edge of a first ring 114 to the rounded beginning edge of the next proximate second ring 114 is at least 0.1 mm, at least 0.15 mm, at least 0.2 mm, at least 0.25 mm, at least 0.3 mm, at least 0.35 mm, at least 0.4 mm, at least 0.45 mm, at least 0.5 mm, at least 0.55 mm, at least 0.6 mm, at least 0.65 mm, at least 0.7 mm, at least 0.75 mm, at least 0.8 mm, at least 0.85 mm, at least 0.9 mm, at least 0.95 mm, at least 1 mm, at least 1.05 mm, at least 1.1 mm, at least 1.15 mm, at least 1.2 mm, at least 1.25 mm, at least 1.3 mm, at least 1.35 mm, at least 1.4 mm, at least 1.45 mm, at least 1.5 mm, at least 1.55 mm, at least 1.6 mm, at least 1.65 mm, at least 1.7 mm, at least 1.75 mm, at least 1.8 mm, at least 1.85 mm, at least 1.9 mm, at least 1.95 mm, at least 2 mm, at least 2.05 mm, at least 2.1 mm, at least 2.15 mm, at least 2.2 mm, at least 2.25 mm, at least 2.3 mm, at least 2.35 mm, at least 2.4 mm, at least 2.45 mm, at least 2.5 mm, at least 2.55 mm, at least 2.6 mm, at least 2.65 mm, at least 2.7 mm, at least 2.75 mm, at least 2.8 mm, at least 2.85 mm, at least 2.9 mm, at least 2.95 mm, or at least 3 mm. In some embodiments, the spacing between the rounded edges of sequential pair of rings 114 is the same for all pairs in the head 102. In some embodiments, the spacing between the rounded edges of each sequential pair of rings 114 is different for at least one of the pairs in the head 102; the head 102 can comprise any combination of different (e.g., at least 2, at least 3, at least 4, at least 5) rounded ring edge spacing distances.

In some embodiments, the distance between the rounded end edge of a first ring 114 to the rounded beginning edge of the next proximate second ring 114 is at most 0.1 mm, at most 0.15 mm, at most 0.2 mm, at most 0.25 mm, at most 0.3 mm, at most 0.35 mm, at most 0.4 mm, at most 0.45 mm, at most 0.5 mm, at most 0.55 mm, at most 0.6 mm, at most 0.65 mm, at most 0.7 mm, at most 0.75 mm, at most 0.8 mm, at most 0.85 mm, at most 0.9 mm, at most 0.95 mm, at most 1 mm, at most 1.05 mm, at most 1.1 mm, at most 1.15 mm, at most 1.2 mm, at most 1.25 mm, at most 1.3 mm, at most 1.35 mm, at most 1.4 mm, at most 1.45 mm, at most 1.5 mm, at most 1.55 mm, at most 1.6 mm, at most 1.65 mm, at most 1.7 mm, at most 1.75 mm, at most 1.8 mm, at most 1.85 mm, at most 1.9 mm, at most 1.95 mm, at most 2 mm, at most 2.05 mm, at most 2.1 mm, at most 2.15 mm, at most 2.2 mm, at most 2.25 mm, at most 2.3 mm, at most 2.35 mm, at most 2.4 mm, at most 2.45 mm, at most 2.5 mm, at most 2.55 mm, at most 2.6 mm, at most 2.65 mm, at most 2.7 mm, at most 2.75 mm, at most 2.8 mm, at most 2.85 mm, at most 2.9 mm, at most 2.95 mm, or at most 3 mm.

In some embodiments, at least one ring of the plurality of rings 114 is an incomplete ring 115 (see e.g., FIG. 2B), e.g., has a missing portion. In some embodiments, the at least one incomplete ring 115 can be included for a swab 100 that is injection molded or otherwise molded. In some embodiments, the at least one incomplete ring 115 can be a site for ejection pins to eject the molded swab 100 from the mold. In some embodiments, the at least one incomplete ring 115 is recessed so as to not result in abrasive or sharp features that would otherwise be introduced into the swab 100 during ejection from the mold; such abrasive or sharp features are disadvantageous as they can directly press against and irritate the nasal cavity. In some embodiments, the at least one incomplete ring 115 allows the remainder of the sample collection head 102 and swab 100 to be very smooth and avoid damage to patients. In some embodiments, the cross-section of the incomplete ring 115 is a semicircle, a truncated circle, or a circle with one or more flat sides. In some embodiments, the incomplete ring 115 does not comprise at least 5%, at least 10%, at least 15%, at least 20%, at least 25%, at least 30%, at least 35%, at least 40%, at least 45%, or at least 50% of a complete ring. In some embodiments, the incomplete ring 115 exposes at least a portion of the axial shaft 103. In some embodiments, the 1^{st}, 2^{nd}, 3^{rd}, 4^{th}, 5^{th}, 6^{th}, 7^{th}, 8^{th}, 9^{th}, and/or 10^{th}, etc. ring 114 (e.g., counting from the head end of the swab 100) is an incomplete ring 115. In some embodiments, the third ring 114 (e.g., counting from the head end of the swab 100) is an incomplete ring 115.

In some embodiments, each incomplete ring 115 exposes the axial shaft 103 of the head 102 for a distance of about 1.5 mm. In some embodiments, each incomplete ring 115 exposes the axial shaft 103 of the head 102 for a distance of at least 0.1 mm, at least 0.15 mm, at least 0.2 mm, at least 0.25 mm, at least 0.3 mm, at least 0.35 mm, at least 0.4 mm, at least 0.45 mm, at least 0.5 mm, at least 0.55 mm, at least 0.6 mm, at least 0.65 mm, at least 0.7 mm, at least 0.75 mm, at least 0.8 mm, at least 0.85 mm, at least 0.9 mm, at least 0.95 mm, at least 1 mm, at least 1.05 mm, at least 1.1 mm, at least 1.15 mm, at least 1.2 mm, at least 1.25 mm, at least 1.3 mm, at least 1.35 mm, at least 1.4 mm, at least 1.45 mm, at least 1.5 mm, at least 1.55 mm, at least 1.6 mm, at least 1.65 mm, at least 1.7 mm, at least 1.75 mm, at least 1.8 mm, at least 1.85 mm, at least 1.9 mm, at least 1.95 mm, at least 2 mm, at least 2.05 mm, at least 2.1 mm, at least 2.15 mm, at least 2.2 mm, at least 2.25 mm, at least 2.3 mm, at least 2.35 mm, at least 2.4 mm, at least 2.45 mm, at least 2.5 mm, at least 2.55 mm, at least 2.6 mm, at least 2.65 mm, at least 2.7 mm, at least 2.75 mm, at least 2.8 mm, at least 2.85 mm, at least 2.9 mm, at least 2.95 mm, at least 3 mm, at least 3.05 mm, at least 3.1 mm, at least 3.15 mm, at least 3.2 mm, at least 3.25 mm, at least 3.3 mm, at least 3.35 mm, at least 3.4 mm, at least 3.45 mm, at least 3.5 mm, at least 3.55 mm, at least 3.6 mm, at least 3.65 mm, at least 3.7 mm, at least 3.75 mm, at least 3.8 mm, at least 3.85 mm, at least 3.9 mm, at least 3.95 mm, or at least 4.0 mm.

In some embodiments, each incomplete ring 115 exposes the axial shaft 103 of the head 102 for a distance of at most 0.1 mm, at most 0.15 mm, at most 0.2 mm, at most 0.25 mm, at most 0.3 mm, at most 0.35 mm, at most 0.4 mm, at most 0.45 mm, at most 0.5 mm, at most 0.55 mm, at most 0.6 mm, at most 0.65 mm, at most 0.7 mm, at most 0.75 mm, at most 0.8 mm, at most 0.85 mm, at most 0.9 mm, at most 0.95 mm, at most 1 mm, at most 1.05 mm, at most 1.1 mm, at most 1.15 mm, at most 1.2 mm, at most 1.25 mm, at most 1.3 mm, at most 1.35 mm, at most 1.4 mm, at most 1.45 mm, at most 1.5 mm, at most 1.55 mm, at most 1.6 mm, at most 1.65 mm, at most 1.7 mm, at most 1.75 mm, at most 1.8 mm, at most 1.85 mm, at most 1.9 mm, at most 1.95 mm, at most 2 mm, at most 2.05 mm, at most 2.1 mm, at most 2.15 mm, at most 2.2 mm, at most 2.25 mm, at most 2.3 mm, at most 2.35 mm, at most 2.4 mm, at most 2.45 mm, at most 2.5 mm, at most 2.55 mm, at most 2.6 mm, at most 2.65 mm, at most 2.7 mm, at most 2.75 mm, at most 2.8 mm, at most 2.85 mm, at most 2.9 mm, at most 2.95 mm, at most 3 mm, at most 3.05 mm, at most 3.1 mm, at most 3.15 mm, at most 3.2 mm, at most 3.25 mm, at most 3.3 mm, at most 3.35 mm, at most 3.4 mm, at most 3.45 mm, at most 3.5 mm, at most 3.55 mm, at most 3.6 mm, at most 3.65 mm, at most 3.7 mm, at most 3.75 mm, at most 3.8 mm, at most 3.85 mm, at most 3.9 mm, at most 3.95 mm, or at most 4.0 mm.

In some embodiments, the distal end of the head 102 (e.g., farthest from the neck 104 and/or handle 106) is tipped with a bulb 101, e.g., to facilitate insertion into the nasal cavity. In some embodiments, the bulb 101 is a sphere or a partial sphere. In some embodiments, the bulb 101 is a hemisphere. In some embodiments, the bulb 101 is an ellipsoid (e.g., a deformed sphere, e.g., a flattened or lengthened sphere) or a partial ellipsoid. In some embodiments, the bulb 101 has a thickness (e.g., the distance from the proximal end of the bulb 101 (e.g., end with the maximum diameter in the case of a hemisphere) to the distal end of the bulb 101) of 1.5 mm. In some embodiments, the bulb 101 has a thickness of 0.1 mm-3.0 mm. In some embodiments, the bulb 101 has a thickness of at least 0.1 mm, at least 0.15 mm, at least 0.2 mm, at least 0.25 mm, at least 0.3 mm, at least 0.35 mm, at least 0.4 mm, at least 0.45 mm, at least 0.5 mm, at least 0.55 mm, at least 0.6 mm, at least 0.65 mm, at least 0.7 mm, at least 0.75 mm, at least 0.8 mm, at least 0.85 mm, at least 0.9 mm, at least 0.95 mm, at least 1 mm, at least 1.05 mm, at least 1.1 mm, at least 1.15 mm, at least 1.2 mm, at least 1.25 mm, at least 1.3 mm, at least 1.35 mm, at least 1.4 mm, at least 1.45 mm, at least 1.5 mm, at least 1.55 mm, at least 1.6 mm, at least 1.65 mm, at least 1.7 mm, at least 1.75 mm, at least 1.8 mm, at least 1.85 mm, at least 1.9 mm, at least 1.95 mm, at least 2 mm, at least 2.05 mm, at least 2.1 mm, at least 2.15 mm, at least 2.2 mm, at least 2.25 mm, at least 2.3 mm, at least 2.35 mm, at least 2.4 mm, at least 2.45 mm, at least 2.5 mm, at least 2.55 mm, at least 2.6 mm, at least 2.65 mm, at least 2.7 mm, at least 2.75 mm, at least 2.8 mm, at least 2.85 mm, at least 2.9 mm, at least 2.95 mm, or at least 3 mm.

In some embodiments, the bulb 101 has a thickness of at most 0.1 mm, at most 0.15 mm, at most 0.2 mm, at most 0.25 mm, at most 0.3 mm, at most 0.35 mm, at most 0.4 mm, at most 0.45 mm, at most 0.5 mm, at most 0.55 mm, at most 0.6 mm, at most 0.65 mm, at most 0.7 mm, at most 0.75 mm, at most 0.8 mm, at most 0.85 mm, at most 0.9 mm, at most 0.95 mm, at most 1 mm, at most 1.05 mm, at most 1.1 mm, at most 1.15 mm, at most 1.2 mm, at most 1.25 mm, at most 1.3 mm, at most 1.35 mm, at most 1.4 mm, at most 1.45 mm, at most 1.5 mm, at most 1.55 mm, at most 1.6 mm, at most 1.65 mm, at most 1.7 mm, at most 1.75 mm, at most 1.8 mm, at most 1.85 mm, at most 1.9 mm, at most 1.95 mm, at most 2 mm, at most 2.05 mm, at most 2.1 mm, at most 2.15 mm, at most 2.2 mm, at most 2.25 mm, at most 2.3 mm, at most 2.35 mm, at most 2.4 mm, at most 2.45 mm, at most 2.5 mm, at most 2.55 mm, at most 2.6 mm, at most 2.65 mm, at most 2.7 mm, at most 2.75 mm, at most 2.8 mm, at most 2.85 mm, at most 2.9 mm, at most 2.95 mm, or at most 3 mm.

In some embodiments, the bulb 101 has a maximum diameter (e.g., closest to the next proximate ring) of 1.0 mm-4.0 mm. In some embodiments, the bulb 101 has a maximum diameter of 2.5 mm. In some embodiments, the bulb 101 has a maximum diameter of 1.0 mm. In some embodiments, the bulb 101 has a maximum diameter of at least 1 mm, at least 1.05 mm, at least 1.1 mm, at least 1.15 mm, at least 1.2 mm, at least 1.25 mm, at least 1.3 mm, at least 1.35 mm, at least 1.4 mm, at least 1.45 mm, at least 1.5 mm, at least 1.55 mm, at least 1.6 mm, at least 1.65 mm, at least 1.7 mm, at least 1.75 mm, at least 1.8 mm, at least 1.85 mm, at least 1.9 mm, at least 1.95 mm, at least 2 mm, at least 2.05 mm, at least 2.1 mm, at least 2.15 mm, at least 2.2 mm, at least 2.25 mm, at least 2.3 mm, at least 2.35 mm, at least 2.4 mm, at least 2.45 mm, at least 2.5 mm, at least 2.55 mm, at least 2.6 mm, at least 2.65 mm, at least 2.7 mm, at least 2.75 mm, at least 2.8 mm, at least 2.85 mm, at least 2.9 mm, at least 2.95 mm, at least 3 mm, at least 3.05 mm, at least 3.1 mm, at least 3.15 mm, at least 3.2 mm, at least 3.25 mm, at least 3.3 mm, at least 3.35 mm, at least 3.4 mm, at least 3.45 mm, at least 3.5 mm, at least 3.55 mm, at least 3.6 mm, at least 3.65 mm, at least 3.7 mm, at least 3.75 mm, at least 3.8 mm, at least 3.85 mm, at least 3.9 mm, at least 3.95 mm, or at least 4.0 mm.

In some embodiments, the bulb 101 has a maximum diameter of at most 1 mm, at most 1.05 mm, at most 1.1 mm, at most 1.15 mm, at most 1.2 mm, at most 1.25 mm, at most 1.3 mm, at most 1.35 mm, at most 1.4 mm, at most 1.45 mm, at most 1.5 mm, at most 1.55 mm, at most 1.6 mm, at most 1.65 mm, at most 1.7 mm, at most 1.75 mm, at most 1.8 mm, at most 1.85 mm, at most 1.9 mm, at most 1.95 mm, at most 2 mm, at most 2.05 mm, at most 2.1 mm, at most 2.15 mm, at most 2.2 mm, at most 2.25 mm, at most 2.3 mm, at most 2.35 mm, at most 2.4 mm, at most 2.45 mm, at most 2.5 mm, at most 2.55 mm, at most 2.6 mm, at most 2.65 mm, at most 2.7 mm, at most 2.75 mm, at most 2.8 mm, at most 2.85 mm, at most 2.9 mm, at most 2.95 mm, at most 3 mm, at most 3.05 mm, at most 3.1 mm, at most 3.15 mm, at most 3.2 mm, at most 3.25 mm, at most 3.3 mm, at most 3.35 mm, at most 3.4 mm, at most 3.45 mm, at most 3.5 mm, at most 3.55 mm, at most 3.6 mm, at most 3.65 mm, at most 3.7 mm, at most 3.75 mm, at most 3.8 mm, at most 3.85 mm, at most 3.9 mm, at most 3.95 mm, or at least most 4.0 mm. In some embodiments, the maximum diameter of the bulb 101 is the same as the diameter of the next proximate ring. In some embodiments, the maximum diameter of the bulb 101 is greater than the diameter of the next proximate ring. In some embodiments, the maximum diameter of the bulb 101 is less than the diameter of the next proximate ring.

In some embodiments, a portion of the axial shaft 103 connects the bulb 101 to the next proximate ring. In embodiments comprising a hemispherical bulb 101, the bulb 101 has a rounded edge (e.g., the edge with the maximum diameter or closest to the next proximate ring). In some embodiments, the distance between the rounded end edge of the bulb 101 to the rounded beginning edge of the next proximate second ring is at least 0.75 mm. In some embodiments, the distance between the rounded end edge of the bulb 101 to the rounded beginning edge of the next proximate second ring is 0.86 mm. In some embodiments, the distance between the rounded end edge of the bulb 101 to the rounded beginning edge of the next proximate second ring is at least 0.1 mm, at least 0.15 mm, at least 0.2 mm, at least 0.25 mm, at least 0.3 mm, at least 0.35 mm, at least 0.4 mm, at least 0.45 mm, at least 0.5 mm, at least 0.55 mm, at least 0.6 mm, at least 0.65 mm, at least 0.7 mm, at least 0.75 mm, at least 0.8 mm, at least 0.85 mm, at least 0.9 mm, at least 0.95 mm, at least 1 mm, at least 1.05 mm, at least 1.1 mm, at least 1.15 mm, at least 1.2 mm, at least 1.25 mm, at least 1.3 mm, at least 1.35 mm, at least 1.4 mm, at least 1.45 mm, at least 1.5 mm, at least 1.55 mm, at least 1.6 mm, at least 1.65 mm, at least 1.7 mm, at least 1.75 mm, at least 1.8 mm, at least 1.85 mm, at least 1.9 mm, at least 1.95 mm, at least 2 mm, at least 2.05 mm, at least 2.1 mm, at least 2.15 mm, at least 2.2 mm, at least 2.25 mm, at least 2.3 mm, at least 2.35 mm, at least 2.4 mm, at least 2.45 mm, at least 2.5 mm, at least 2.55 mm, at least 2.6 mm, at least 2.65 mm, at least 2.7 mm, at least 2.75 mm, at least 2.8 mm, at least 2.85 mm, at least 2.9 mm, at least 2.95 mm, or at least 3 mm.

In some embodiments, the distance between the rounded end edge of the bulb 101 to the rounded beginning edge of the next proximate second ring is at most 0.1 mm, at most 0.15 mm, at most 0.2 mm, at most 0.25 mm, at most 0.3 mm, at most 0.35 mm, at most 0.4 mm, at most 0.45 mm, at most 0.5 mm, at most 0.55 mm, at most 0.6 mm, at most 0.65 mm, at most 0.7 mm, at most 0.75 mm, at most 0.8 mm, at most 0.85 mm, at most 0.9 mm, at most 0.95 mm, at most 1 mm, at most 1.05 mm, at most 1.1 mm, at most 1.15 mm, at most 1.2 mm, at most 1.25 mm, at most 1.3 mm, at most 1.35 mm, at most 1.4 mm, at most 1.45 mm, at most 1.5 mm, at most 1.55 mm, at most 1.6 mm, at most 1.65 mm, at most 1.7 mm, at most 1.75 mm, at most 1.8 mm, at most 1.85 mm, at most 1.9 mm, at most 1.95 mm, at most 2 mm, at most 2.05 mm, at most 2.1 mm, at most 2.15 mm, at most 2.2 mm, at most 2.25 mm, at most 2.3 mm, at most 2.35 mm, at most 2.4 mm, at most 2.45 mm, at most 2.5 mm, at most 2.55 mm, at most 2.6 mm, at most 2.65 mm, at most 2.7 mm, at most 2.75 mm, at most 2.8 mm, at most 2.85 mm, at most 2.9 mm, at most 2.95 mm, or at most 3 mm.

In some embodiments, the spacing between the rounded edges of the bulb 101 and the next proximate ring is the same as the spacing between the rounded edges of the plurality of rings 114. In some embodiments, the spacing between the rounded edges of the bulb 101 and the next proximate ring is different from the spacing between the rounded edges of the plurality of rings 114. In some embodiments, the spacing between the rounded edges of the bulb 101 and the next proximate ring is less than the spacing between the rounded edges of the plurality of rings 114. In some embodiments, the spacing between the rounded edges of the bulb 101 and the next proximate ring is greater than the spacing between the rounded edges of the plurality of rings 114.

In some embodiments, the sample collection head 102 comprises a spiral axis groove, e.g., a depression of similar dimensions to the rings 114 disclosed herein that spirals around the axial shaft 103 of the head. In some embodiments, the sample collection head comprises a spiral axis flange, e.g., an elevation or protrusion of similar dimensions to the rings 114 disclosed herein that spirals around the axial shaft 103 of the head 102. In some embodiments, the spiral axis groove or spiral axis flange is spaced 0.1 mm-3 mm apart. In some embodiments, the spiral axis groove or spiral axis flange is spaced 0.75 mm apart. In some embodiments, the spiral axis groove or spiral axis flange has a thickness of 0.1 mm-3 mm. In some embodiments, the spiral axis groove or spiral axis flange has a thickness of 1.0 mm. In some embodiments, the spiral axis groove or spiral axis flange has a diameter of 1.0 mm-4.0 mm. In some embodiments, the spiral axis groove or spiral axis flange has a diameter of 2.5 mm. In some embodiments, the spiral axis groove or spiral axis flange are tapered. In some embodiments, the spiral axis groove or spiral axis flange has rounded edges. In some embodiments, the sample collection head 102 comprises any combination of a plurality of spaced annular rings 114, a spiral axis groove, or spiral axis flange. In some embodiments, the sample collection head 102 comprises a plurality of rings 114 and a spiral axis groove. In some embodiments, the sample collection head 102 comprises a plurality of rings 114 and a spiral axis flange. In some embodiments, the sample collection head 102 comprises a spiral axis groove and a spiral axis flange. In some embodiments, the sample collection head 102 comprises a plurality of rings 114, a spiral axis groove, and a spiral axis flange. In some embodiments, the sample collection head 102 comprises a plurality of spiral axis grooves or a plurality of spiral axis flanges, e.g., 2, 3, 4, 5, 6, 7, 8, 9, 10, 11, 12, 13, 14, 15, 16, 17, 18, 19, 20, 21, 22, 23, 24, 25, or more spiral axis grooves or spiral axis flanges. In some embodiments, the plurality of spiral axis grooves or the plurality of spiral axis flanges are continuous or discontinuous, with the same or different spacing, thickness, and/or diameter.

In some embodiments, the head 102 does not comprise a fibrous coating. As used herein, the term “fibrous material” refers to a plurality of discrete fibers. The fibers can be plant-derived or animal-derived, synthetic, or some combination of these. In plant-derived fibrous materials, the fibers are at least predominantly of plant origin, non-limiting examples of which include cotton, wood, papyrus, rice, ficus, mulberry, yucca, sisal, bowstring hemp, and New Zealand flax. Additional non-limiting examples of fibrous coatings that can be found in traditional swabs include cotton, cellulose, rayon, and polyester.

In some embodiments, the head 102 is stippled, roughened, or textured. As used herein, the term “stipple” means to mark or engrave a surface with number small dots or specks. As used herein, the term “roughen” means to cause to have an uneven, irregular, non-smooth surface, e.g., through abrasion. As used herein, the term “texture” means to cause to have a rough or raised or engraved surface. The texture can comprise a regular or repeated pattern (e.g., parallel grooves, perpendicular grooves, circles such as concentric circles, etc.) or an irregular non-patterned configuration, or any combination of regular and irregular textures. In some embodiments, the texture can comprise nanotexture, e.g., with dimensions (e.g., depth, thickness, and/or length) ranging from 1 nm-100 µm (e.g., at least 1 nm, at least 10 nm, at least 100 nm, at least 1 µm, at least 10 µm, at least 20 µm, at least 30 µm, at least 40 µm, at least 50 µm, at least 60 µm, at least 70 µm, at least 80 µm, at least 90 µm, or at least 100 µm). In some embodiments, the stippling, roughening, or texturing is applied using bead-blasting. In some embodiments, the stippling, roughening, or texturing of the head 102 increases the surface area of the head 102 by at least 1%, at least 5%, at least 10%, at least 20%, at least 30%, at least 40%, at least 50%, at least 60%, at least 70%, at least 80, at least 90%, at least 100%, at least 150%, at least 200%, at least 250%, at least 300%, at least 350%, at least 400%, at least 450%, or at least 500%.

In some embodiments, the length of the sample collection head 102 (e.g., the “proximal” end of the head 102, e.g., the first ring of the plurality of rings 114, to the from “distal” end of the head 102, e.g., the termination of the head 102 at the bulb 101) is at least 15 mm. In some embodiments, the length of the head 102 is about 19 mm. In some embodiments, the length of the head 102 is at least 5 mm, at least 5.5 mm, at least 6 mm, at least 6.5 mm, at least 7 mm, at least 7.5 mm, at least 8 mm, at least 8.5 mm, at least 9 mm, at least 9.5 mm, at least 10 mm, at least 10.5 mm, at least 11 mm, at least 11.5 mm, at least 12 mm, at least 12.5 mm, at least 13 mm, at least 13.5 mm, at least 14 mm, at least 14.5 mm, at least 15 mm, at least 15.5 mm, at least 16 mm, at least 16.5 mm, at least 17 mm, at least 17.5 mm, at least 18 mm, at least 18.5 mm, at least 19 mm, at least 19.5 mm, at least 20 mm, at least 20.5 mm, at least 21 mm, at least 21.5 mm, at least 22 mm, at least 22.5 mm, at least 23 mm, at least 23.5 mm, at least 24 mm, at least 24.5 mm, at least 25 mm, at least 25.5 mm, at least 26 mm, at least 26.5 mm, at least 27 mm, at least 27.5 mm, at least 28 mm, at least 28.5 mm, at least 29 mm, at least 29.5 mm, at least 30 mm, at least 30.5 mm, at least 31 mm, at least 31.5 mm, at least 32 mm, at least 32.5 mm, at least 33 mm, at least 33.5 mm, at least 34 mm, at least 34.5 mm, at least 35 mm, at least 35.5 mm, at least 36 mm, at least 36.5 mm, at least 37 mm, at least 37.5 mm, at least 38 mm, at least 38.5 mm, at least 39 mm, at least 39.5 mm, at least 40 mm, at least 40.5 mm, at least 41 mm, at least 41.5 mm, at least 42 mm, at least 42.5 mm, at least 43 mm, at least 43.5 mm, at least 44 mm, at least 44.5 mm, at least 45 mm, at least 45.5 mm, at least 46 mm, at least 46.5 mm, at least 47 mm, at least 47.5 mm, at least 48 mm, at least 48.5 mm, at least 49 mm, at least 49.5 mm, or at least 50 mm.

In some embodiments, the length of the head 102 is at most 5 mm, at most 5.5 mm, at most 6 mm, at most 6.5 mm, at most 7 mm, at most 7.5 mm, at most 8 mm, at most 8.5 mm, at most 9 mm, at most 9.5 mm, at most 10 mm, at most 10.5 mm, at most 11 mm, at most 11.5 mm, at most 12 mm, at most 12.5 mm, at most 13 mm, at most 13.5 mm, at most 14 mm, at most 14.5 mm, at most 15 mm, at most 15.5 mm, at most 16 mm, at most 16.5 mm, at most 17 mm, at most 17.5 mm, at most 18 mm, at most 18.5 mm, at most 19 mm, at most 19.5 mm, at most 20 mm, at most 20.5 mm, at most 21 mm, at most 21.5 mm, at most 22 mm, at most 22.5 mm, at most 23 mm, at most 23.5 mm, at most 24 mm, at most 24.5 mm, at most 25 mm, at most 25.5 mm, at most 26 mm, at most 26.5 mm, at most 27 mm, at most 27.5 mm, at most 28 mm, at most 28.5 mm, at most 29 mm, at most 29.5 mm, at most 30 mm, at most 30.5 mm, at most 31 mm, at most 31.5 mm, at most 32 mm, at most 32.5 mm, at most 33 mm, at most 33.5 mm, at most 34 mm, at most 34.5 mm, at most 35 mm, at most 35.5 mm, at most 36 mm, at most 36.5 mm, at most 37 mm, at most 37.5 mm, at most 38 mm, at most 38.5 mm, at most 39 mm, at most 39.5 mm, at most 40 mm, at most 40.5 mm, at most 41 mm, at most 41.5 mm, at most 42 mm, at most 42.5 mm, at most 43 mm, at most 43.5 mm, at most 44 mm, at most 44.5 mm, at most 45 mm, at most 45.5 mm, at most 46 mm, at most 46.5 mm, at most 47 mm, at most 47.5 mm, at most 48 mm, at most 48.5 mm, at most 49 mm, at most 49.5 mm, or at most 50 mm. In some embodiments of any of the aspects, the combined length of the head 102 and the neck 104 is at most 25 mm, at most 30 mm, at most 35 mm, at most 40 mm, at most 45 mm, at most 50 mm, at most 55 mm, at most 60 mm, at most 65 mm, at most 70 mm, at most 75 mm, at most 80 mm, at most 85 mm, at most 90 mm, at most 95 mm, at most 100 mm, at most 105 mm, at most 110 mm, at most 115 mm, at most 120 mm, at most 125 mm, at most 130 mm, at most 135 mm, at most 140 mm, at most 145 mm, or at most 150 mm.

### Neck

In some embodiments, the swab 100 further comprises a neck 104. In some embodiments, the neck 104 connects the sample collection head 102 to the handle 106. In some embodiments, the neck 104 comprises a rod (see e.g., FIG. 1). In some embodiments, the cross-section of the neck 104 is a circle, a semicircle, a truncated circle, or a circle with one or more flat sides. In some embodiments, the cross-section of the neck 104 is a circle. In some embodiments, the neck 104 comprises a cylindrical rod. In some embodiments, the neck 104 comprises a rod with a polygonal cross section, e.g., a cross-section in the shape of a triangle, a square, a quadrilateral, a trapezoid, a pentagon, a hexagon, or a polygon with at least 7, 8, 9, 10, 11, 12, 13, 14, 15, 16, 17, 18, 19, 20 or more sides. In some embodiments, at least one side of the cross-section of the neck 104 comprises a convex and/or concave curve. In some embodiments, the cross section of the neck 104 is a rotationally symmetric shape. In some embodiments, the cross section of the neck 104 is an asymmetric shape. In some embodiments, the neck cross-section is the same for the entirety of the neck 104. In some embodiments, the neck cross-section is different for at least one portion of the neck 104; the neck 104 can comprise any combination of different (e.g., at least 2, at least 3, at least 4, at least 5) neck cross-sections. In some embodiments, the neck 104 tapers from a maximum diameter (e.g., towards the handle 106) to a smaller diameter (e.g., towards the head 102). In some embodiments, the maximum diameter of the neck 104 is the same as the minimum diameter of the handle 106. In some embodiments, the maximum diameter of the neck 104 is less than the minimum diameter of the handle 106. In some embodiments, the maximum diameter of the neck 104 is greater than the minimum diameter of the handle 106. In some embodiments, the minimum diameter of the neck 104 is the same as the maximum diameter of the axial shaft 103 of the sample collection head 102. In some embodiments, the minimum diameter of the neck 104 is less than as the maximum diameter of the axial shaft 103 of the sample collection head 102. In some embodiments, the minimum diameter of the neck 104 is greater than the maximum diameter of the axial shaft 103 of the sample collection head 102.

In some embodiments, the rate of the tapering of the neck 104 is constant and/or continuous. In some embodiments, the rate of the tapering of the neck 104 is non-constant and/or discontinuous. In some embodiments, the neck 104 comprises a plurality of sections (e.g., 2, 3, 4, 5, 6, 7, 8, 9, 10 or more) each with a different rate of tapering and/or no tapering. In some embodiments, each section of the neck 104 is continuous with the next proximate section, e.g., a first section (farther from the head 102) of the neck 104 has a minimum diameter that is the same as the maximum diameter of the next proximate second section (closer to the head 102) of the neck 104.

In some embodiments, the neck 104 (or any section of the neck 104) has a maximum diameter (e.g., towards the handle 106) of about 1.0 mm-4.0 mm. In some embodiments, the neck 104 (or any section of the neck 104) has a maximum diameter of about 1.5 mm. In some embodiments, the neck 104 (or any section of the neck 104) has a maximum diameter of at least 1 mm, at least 1.05 mm, at least 1.1 mm, at least 1.15 mm, at least 1.2 mm, at least 1.25 mm, at least 1.3 mm, at least 1.35 mm, at least 1.4 mm, at least 1.45 mm, at least 1.5 mm, at least 1.55 mm, at least 1.6 mm, at least 1.65 mm, at least 1.7 mm, at least 1.75 mm, at least 1.8 mm, at least 1.85 mm, at least 1.9 mm, at least 1.95 mm, at least 2 mm, at least 2.05 mm, at least 2.1 mm, at least 2.15 mm, at least 2.2 mm, at least 2.25 mm, at least 2.3 mm, at least 2.35 mm, at least 2.4 mm, at least 2.45 mm, at least 2.5 mm, at least 2.55 mm, at least 2.6 mm, at least 2.65 mm, at least 2.7 mm, at least 2.75 mm, at least 2.8 mm, at least 2.85 mm, at least 2.9 mm, at least 2.95 mm, at least 3 mm, at least 3.05 mm, at least 3.1 mm, at least 3.15 mm, at least 3.2 mm, at least 3.25 mm, at least 3.3 mm, at least 3.35 mm, at least 3.4 mm, at least 3.45 mm, at least 3.5 mm, at least 3.55 mm, at least 3.6 mm, at least 3.65 mm, at least 3.7 mm, at least 3.75 mm, at least 3.8 mm, at least 3.85 mm, at least 3.9 mm, at least 3.95 mm, or at least 4.0 mm.

In some embodiments, the neck 104 (or any section of the neck 104) has a maximum diameter of at most 1 mm, at most 1.05 mm, at most 1.1 mm, at most 1.15 mm, at most 1.2 mm, at most 1.25 mm, at most 1.3 mm, at most 1.35 mm, at most 1.4 mm, at most 1.45 mm, at most 1.5 mm, at most 1.55 mm, at most 1.6 mm, at most 1.65 mm, at most 1.7 mm, at most 1.75 mm, at most 1.8 mm, at most 1.85 mm, at most 1.9 mm, at most 1.95 mm, at most 2 mm, at most 2.05 mm, at most 2.1 mm, at most 2.15 mm, at most 2.2 mm, at most 2.25 mm, at most 2.3 mm, at most 2.35 mm, at most 2.4 mm, at most 2.45 mm, at most 2.5 mm, at most 2.55 mm, at most 2.6 mm, at most 2.65 mm, at most 2.7 mm, at most 2.75 mm, at most 2.8 mm, at most 2.85 mm, at most 2.9 mm, at most 2.95 mm, at most 3 mm, at most 3.05 mm, at most 3.1 mm, at most 3.15 mm, at most 3.2 mm, at most 3.25 mm, at most 3.3 mm, at most 3.35 mm, at most 3.4 mm, at most 3.45 mm, at most 3.5 mm, at most 3.55 mm, at most 3.6 mm, at most 3.65 mm, at most 3.7 mm, at most 3.75 mm, at most 3.8 mm, at most 3.85 mm, at most 3.9 mm, at most 3.95 mm, or at most 4.0 mm.

In some embodiments, the neck 104 (or any section of the neck 104) has a minimum diameter (e.g., towards the head 102) of about 0.5 mm-3.5 mm. In some embodiments, the neck 104 (or any section of the neck 104) has a minimum diameter of 1.2 mm. In some embodiments, the neck 104 (or any section of the neck 104) has a minimum diameter of at least 0.5 mm, at least 0.55 mm, at least 0.6 mm, at least 0.65 mm, at least 0.7 mm, at least 0.75 mm, at least 0.8 mm, at least 0.85 mm, at least 0.9 mm, at least 0.95 mm, at least 1 mm, at least 1.05 mm, at least 1.1 mm, at least 1.15 mm, at least 1.2 mm, at least 1.25 mm, at least 1.3 mm, at least 1.35 mm, at least 1.4 mm, at least 1.45 mm, at least 1.5 mm, at least 1.55 mm, at least 1.6 mm, at least 1.65 mm, at least 1.7 mm, at least 1.75 mm, at least 1.8 mm, at least 1.85 mm, at least 1.9 mm, at least 1.95 mm, at least 2 mm, at least 2.05 mm, at least 2.1 mm, at least 2.15 mm, at least 2.2 mm, at least 2.25 mm, at least 2.3 mm, at least 2.35 mm, at least 2.4 mm, at least 2.45 mm, at least 2.5 mm, at least 2.55 mm, at least 2.6 mm, at least 2.65 mm, at least 2.7 mm, at least 2.75 mm, at least 2.8 mm, at least 2.85 mm, at least 2.9 mm, at least 2.95 mm, at least 3 mm, at least 3.05 mm, at least 3.1 mm, at least 3.15 mm, at least 3.2 mm, at least 3.25 mm, at least 3.3 mm, at least 3.35 mm, at least 3.4 mm, at least 3.45 mm, or at least 3.5 mm.

In some embodiments, the neck 104 (or any section of the neck 104) has a minimum diameter of at most 0.5 mm, at most 0.55 mm, at most 0.6 mm, at most 0.65 mm, at most 0.7 mm, at most 0.75 mm, at most 0.8 mm, at most 0.85 mm, at most 0.9 mm, at most 0.95 mm, at most 1 mm, at most 1.05 mm, at most 1.1 mm, at most 1.15 mm, at most 1.2 mm, at most 1.25 mm, at most 1.3 mm, at most 1.35 mm, at most 1.4 mm, at most 1.45 mm, at most 1.5 mm, at most 1.55 mm, at most 1.6 mm, at most 1.65 mm, at most 1.7 mm, at most 1.75 mm, at most 1.8 mm, at most 1.85 mm, at most 1.9 mm, at most 1.95 mm, at most 2 mm, at most 2.05 mm, at most 2.1 mm, at most 2.15 mm, at most 2.2 mm, at most 2.25 mm, at most 2.3 mm, at most 2.35 mm, at most 2.4 mm, at most 2.45 mm, at most 2.5 mm, at most 2.55 mm, at most 2.6 mm, at most 2.65 mm, at most 2.7 mm, at most 2.75 mm, at most 2.8 mm, at most 2.85 mm, at most 2.9 mm, at most 2.95 mm, at most 3 mm, at most 3.05 mm, at most 3.1 mm, at most 3.15 mm, at most 3.2 mm, at most 3.25 mm, at most 3.3 mm, at most 3.35 mm, at most 3.4 mm, at most 3.45 mm, or at most 3.5 mm.

In some embodiments, the length of the neck 104 is about 20 mm-100 mm. In some embodiments (see e.g., FIG. 1), the length of the neck 104 is at least 50 mm. In some embodiments, the length of the neck 104 is about 56.75 mm. In some embodiments (e.g., comprising a plunger 110, see e.g., FIG. 6), the length of the neck 104 is at least 25 mm. In some embodiments, the length of the neck 104 is about 28 mm. In some embodiments of any of the aspects, the length of the neck 104 is at least 20 mm, at least 25 mm, at least 30 mm, at least 35 mm, at least 40 mm, at least 45 mm, at least 50 mm, at least 55 mm, at least 60 mm, at least 65 mm, at least 70 mm, at least 75 mm, at least 80 mm, at least 85 mm, at least 90 mm, at least 95 mm, or at least 100 mm. In some embodiments of any of the aspects, the length of the neck 104 is at most 20 mm, at most 25 mm, at most 30 mm, at most 35 mm, at most 40 mm, at most 45 mm, at most 50 mm, at most 55 mm, at most 60 mm, at most 65 mm, at most 70 mm, at most 75 mm, at most 80 mm, at most 85 mm, at most 90 mm, at most 95 mm, or at most 100 mm.

In some embodiments, the combined length of the head 102 and the neck 104 is about 25 mm-150 mm. In some embodiments (see e.g., FIG. 1), the combined length of the head 102 and the neck 104 is at least 75 mm. In some embodiments, the combined length of the head 102 and the neck 104 is about 75.75 mm. In some embodiments (e.g., comprising a plunger 110, see e.g., FIG. 6), the combined length of the head 102 and the neck 104 is at least 45 mm. In some embodiments, the combined length of the head 102 and the neck 104 is about 47.05 mm. In some embodiments of any of the aspects, the combined length of the head 102 and the neck 104 is at least 25 mm, at least 30 mm, at least 35 mm, at least 40 mm, at least 45 mm, at least 50 mm, at least 55 mm, at least 60 mm, at least 65 mm, at least 70 mm, at least 75 mm, at least 80 mm, at least 85 mm, at least 90 mm, at least 95 mm, at least 100 mm, at least 105 mm, at least 110 mm, at least 115 mm, at least 120 mm, at least 125 mm, at least 130 mm, at least 135 mm, at least 140 mm, at least 145 mm, or at least 150 mm. In some embodiments of any of the aspects, the combined length of the head 102 and the neck 104 is at most 25 mm, at most 30 mm, at most 35 mm, at most 40 mm, at most 45 mm, at most 50 mm, at most 55 mm, at most 60 mm, at most 65 mm, at most 70 mm, at most 75 mm, at most 80 mm, at most 85 mm, at most 90 mm, at most 95 mm, at most 100 mm, at most 105 mm, at most 110 mm, at most 115 mm, at most 120 mm, at most 125 mm, at most 130 mm, at most 135 mm, at most 140 mm, at most 145 mm, or at most 150 mm.

### Handle

In some embodiments, the swab 100 further comprises a handle 106. In some embodiments, the handle 106 connects to neck 104. In some embodiments, the handle 106 comprises a rod (see e.g., FIG. 1). In some embodiments, the cross-section of the handle 106 is a circle, a semicircle, a truncated circle, or a circle with one or more flat sides. In some embodiments, the cross-section of the handle 106 is a circle. In some embodiments, the handle 106 comprises a cylindrical rod. In some embodiments, the handle 106 comprises a rod with a polygonal cross section, e.g., a cross-section in the shape of a triangle, a square, a quadrilateral, a trapezoid, a pentagon, a hexagon, or a polygon with at least 7, 8, 9, 10, 11, 12, 13, 14, 15, 16, 17, 18, 19, 20 or more sides. In some embodiments, at least one side of the cross-section of the handle 106 comprises a convex and/or concave curve. In some embodiments, the cross section of the handle 106 is a rotationally symmetric shape. In some embodiments, the cross section of the handle 106 is an asymmetric shape. In some embodiments, the handle cross-section is the same for the entirety of the handle 106. In some embodiments, the handle cross-section is different for at least one portion of the handle 106; the handle 106 can comprise any combination of different (e.g., at least 2, at least 3, at least 4, at least 5) handle cross-sections. In some embodiments, the handle 106 tapers from a maximum diameter (e.g., towards the proximal end of the handle 106) to a smaller diameter (e.g., towards the head 102 or the distal end of the handle 106). In some embodiments, the minimum diameter of the handle 106 is the same as the maximum diameter of the neck 104. In some embodiments, the minimum diameter of the handle 106 is less than the maximum diameter of the neck 104. In some embodiments, the minimum diameter of the handle 106 is greater than the maximum diameter of the neck 104. In some embodiments, the maximum diameter of the handle 106 is the same as the maximum diameter of the sample collection head 102 (e.g., the maximum dimeter of the annular rings 114 of the sample collection head 102). In some embodiments, the maximum diameter of the handle 106 is greater than the maximum diameter of the sample collection head 102. In some embodiments, the maximum diameter of the handle 106 is less than the maximum diameter of the sample collection head 102.

In some embodiments, the rate of the tapering of the handle 106 is constant and/or continuous. In some embodiments, the rate of the tapering of the handle 106 is non-constant and/or discontinuous. In some embodiments, the handle 106 comprises a plurality of sections (e.g., 2, 3, 4, 5, 6, 7, 8, 9, 10 or more) each with a different rate of tapering and/or no tapering. In some embodiments, each section of the handle 106 is continuous with the next proximate section, e.g., a first section (farther from the head 102) of the neck 104 has a minimum diameter that is the same as the maximum diameter of the next proximate second section (closer to the head 102) of the handle 106.

In some embodiments, the handle 106 has a maximum diameter of about 2.5 mm. In some embodiments, the handle 106 has a maximum diameter of about 1 mm-10mm. In some embodiments, the handle 106 has a maximum diameter of at least 1 mm, at least 1.05 mm, at least 1.1 mm, at least 1.15 mm, at least 1.2 mm, at least 1.25 mm, at least 1.3 mm, at least 1.35 mm, at least 1.4 mm, at least 1.45 mm, at least 1.5 mm, at least 1.55 mm, at least 1.6 mm, at least 1.65 mm, at least 1.7 mm, at least 1.75 mm, at least 1.8 mm, at least 1.85 mm, at least 1.9 mm, at least 1.95 mm, at least 2 mm, at least 2.05 mm, at least 2.1 mm, at least 2.15 mm, at least 2.2 mm, at least 2.25 mm, at least 2.3 mm, at least 2.35 mm, at least 2.4 mm, at least 2.45 mm, at least 2.5 mm, at least 2.55 mm, at least 2.6 mm, at least 2.65 mm, at least 2.7 mm, at least 2.75 mm, at least 2.8 mm, at least 2.85 mm, at least 2.9 mm, at least 2.95 mm, at least 3 mm, at least 3.05 mm, at least 3.1 mm, at least 3.15 mm, at least 3.2 mm, at least 3.25 mm, at least 3.3 mm, at least 3.35 mm, at least 3.4 mm, at least 3.45 mm, at least 3.5 mm, at least 3.55 mm, at least 3.6 mm, at least 3.65 mm, at least 3.7 mm, at least 3.75 mm, at least 3.8 mm, at least 3.85 mm, at least 3.9 mm, at least 3.95 mm, at least 4.0 mm, at least 4.5 mm, at least 5 mm, at least 5.5 mm, at least 6 mm, at least 6.5 mm, at least 7 mm, at least 7.5 mm, at least 8 mm, at least 8.5 mm, at least 9 mm, at least 9.5 mm, or at least 10 mm.

In some embodiments, In some embodiments, the handle 106 has a maximum diameter of at most 1 mm, at most 1.05 mm, at most 1.1 mm, at most 1.15 mm, at most 1.2 mm, at most 1.25 mm, at most 1.3 mm, at most 1.35 mm, at most 1.4 mm, at most 1.45 mm, at most 1.5 mm, at most 1.55 mm, at most 1.6 mm, at most 1.65 mm, at most 1.7 mm, at most 1.75 mm, at most 1.8 mm, at most 1.85 mm, at most 1.9 mm, at most 1.95 mm, at most 2 mm, at most 2.05 mm, at most 2.1 mm, at most 2.15 mm, at most 2.2 mm, at most 2.25 mm, at most 2.3 mm, at most 2.35 mm, at most 2.4 mm, at most 2.45 mm, at most 2.5 mm, at most 2.55 mm, at most 2.6 mm, at most 2.65 mm, at most 2.7 mm, at most 2.75 mm, at most 2.8 mm, at most 2.85 mm, at most 2.9 mm, at most 2.95 mm, at most 3 mm, at most 3.05 mm, at most 3.1 mm, at most 3.15 mm, at most 3.2 mm, at most 3.25 mm, at most 3.3 mm, at most 3.35 mm, at most 3.4 mm, at most 3.45 mm, at most 3.5 mm, at most 3.55 mm, at most 3.6 mm, at most 3.65 mm, at most 3.7 mm, at most 3.75 mm, at most 3.8 mm, at most 3.85 mm, at most 3.9 mm, at most 3.95 mm, at most 4.0 mm, at most 4.5 mm, at most 5 mm, at most 5.5 mm, at most 6 mm, at most 6.5 mm, at most 7 mm, at most 7.5 mm, at most 8 mm, at most 8.5 mm, at most 9 mm, at most 9.5 mm, or at most 10 mm.

In some embodiments, the handle 106 has a minimum diameter of about 1.5 mm. In some embodiments, the handle 106 has a minimum diameter of about 0.5 mm-3 mm. In some embodiments, the handle 106 has a minimum diameter of at least 0.5 mm, at least 0.55 mm, at least 0.6 mm, at least 0.65 mm, at least 0.7 mm, at least 0.75 mm, at least 0.8 mm, at least 0.85 mm, at least 0.9 mm, at least 0.95 mm, at least 1 mm, at least 1.05 mm, at least 1.1 mm, at least 1.15 mm, at least 1.2 mm, at least 1.25 mm, at least 1.3 mm, at least 1.35 mm, at least 1.4 mm, at least 1.45 mm, at least 1.5 mm, at least 1.55 mm, at least 1.6 mm, at least 1.65 mm, at least 1.7 mm, at least 1.75 mm, at least 1.8 mm, at least 1.85 mm, at least 1.9 mm, at least 1.95 mm, at least 2 mm, at least 2.05 mm, at least 2.1 mm, at least 2.15 mm, at least 2.2 mm, at least 2.25 mm, at least 2.3 mm, at least 2.35 mm, at least 2.4 mm, at least 2.45 mm, at least 2.5 mm, at least 2.55 mm, at least 2.6 mm, at least 2.65 mm, at least 2.7 mm, at least 2.75 mm, at least 2.8 mm, at least 2.85 mm, at least 2.9 mm, at least 2.95 mm, or at least 3 mm.

In some embodiments, the handle 106 has a minimum diameter of at most 0.5 mm, at most 0.55 mm, at most 0.6 mm, at most 0.65 mm, at most 0.7 mm, at most 0.75 mm, at most 0.8 mm, at most 0.85 mm, at most 0.9 mm, at most 0.95 mm, at most 1 mm, at most 1.05 mm, at most 1.1 mm, at most 1.15 mm, at most 1.2 mm, at most 1.25 mm, at most 1.3 mm, at most 1.35 mm, at most 1.4 mm, at most 1.45 mm, at most 1.5 mm, at most 1.55 mm, at most 1.6 mm, at most 1.65 mm, at most 1.7 mm, at most 1.75 mm, at most 1.8 mm, at most 1.85 mm, at most 1.9 mm, at most 1.95 mm, at most 2 mm, at most 2.05 mm, at most 2.1 mm, at most 2.15 mm, at most 2.2 mm, at most 2.25 mm, at most 2.3 mm, at most 2.35 mm, at most 2.4 mm, at most 2.45 mm, at most 2.5 mm, at most 2.55 mm, at most 2.6 mm, at most 2.65 mm, at most 2.7 mm, at most 2.75 mm, at most 2.8 mm, at most 2.85 mm, at most 2.9 mm, at most 2.95 mm, or at most 3 mm.

In some embodiments, the handle 106 has a length of about 75 mm. In some embodiments, the handle 106 has a length of about 50 mm-150 mm. In some embodiments, the handle 106 has a length of at least 50 mm, at least 55 mm, at least 60 mm, at least 65 mm, at least 70 mm, at least 75 mm, at least 80 mm, at least 85 mm, at least 90 mm, at least 95 mm, at least 100 mm, at least 105 mm, at least 110 mm, at least 115 mm, at least 120 mm, at least 125 mm, at least 130 mm, at least 135 mm, at least 140 mm, at least 145 mm, or at least 150 mm. In some embodiments, the handle 106 has a length of at most 50 mm, at most 55 mm, at most 60 mm, at most 65 mm, at most 70 mm, at most 75 mm, at most 80 mm, at most 85 mm, at most 90 mm, at most 95 mm, at most 100 mm, at most 105 mm, at most 110 mm, at most 115 mm, at most 120 mm, at most 125 mm, at most 130 mm, at most 135 mm, at most 140 mm, at most 145 mm, or at most 150 mm.

In some embodiments, the proximal end of the handle 106 (e.g., farthest from the head 102) is tipped with a bulb 101. In some embodiments, the bulb 101 is a sphere or a partial sphere. In some embodiments, the bulb 101 is a hemisphere. In some embodiments, the bulb 101 is an ellipsoid (e.g., a deformed sphere, e.g., a flattened or lengthened sphere) or a partial ellipsoid. In some embodiments, the bulb 101 has a thickness (e.g., the distance from the distal end of the bulb 101 (e.g., end with the maximum diameter in the case of a hemisphere) to the proximal end of the bulb 101) of 1.5 mm. In some embodiments, the bulb 101 has a thickness of 0.1 mm-3.0 mm. In some embodiments, the bulb 101 has a thickness of at least 0.1 mm, at least 0.15 mm, at least 0.2 mm, at least 0.25 mm, at least 0.3 mm, at least 0.35 mm, at least 0.4 mm, at least 0.45 mm, at least 0.5 mm, at least 0.55 mm, at least 0.6 mm, at least 0.65 mm, at least 0.7 mm, at least 0.75 mm, at least 0.8 mm, at least 0.85 mm, at least 0.9 mm, at least 0.95 mm, at least 1 mm, at least 1.05 mm, at least 1.1 mm, at least 1.15 mm, at least 1.2 mm, at least 1.25 mm, at least 1.3 mm, at least 1.35 mm, at least 1.4 mm, at least 1.45 mm, at least 1.5 mm, at least 1.55 mm, at least 1.6 mm, at least 1.65 mm, at least 1.7 mm, at least 1.75 mm, at least 1.8 mm, at least 1.85 mm, at least 1.9 mm, at least 1.95 mm, at least 2 mm, at least 2.05 mm, at least 2.1 mm, at least 2.15 mm, at least 2.2 mm, at least 2.25 mm, at least 2.3 mm, at least 2.35 mm, at least 2.4 mm, at least 2.45 mm, at least 2.5 mm, at least 2.55 mm, at least 2.6 mm, at least 2.65 mm, at least 2.7 mm, at least 2.75 mm, at least 2.8 mm, at least 2.85 mm, at least 2.9 mm, at least 2.95 mm, or at least 3 mm.

In some embodiments, the bulb 101 has a maximum diameter (e.g., closest to proximal end of the swab 100) of 1.0 mm-4.0 mm. In some embodiments, the bulb 101 has a maximum diameter of 2.5 mm. In some embodiments, the bulb 101 has a maximum diameter of 1.0 mm. In some embodiments, the bulb 101 has a maximum diameter of at most 1 mm, at most 1.05 mm, at most 1.1 mm, at most 1.15 mm, at most 1.2 mm, at most 1.25 mm, at most 1.3 mm, at most 1.35 mm, at most 1.4 mm, at most 1.45 mm, at most 1.5 mm, at most 1.55 mm, at most 1.6 mm, at most 1.65 mm, at most 1.7 mm, at most 1.75 mm, at most 1.8 mm, at most 1.85 mm, at most 1.9 mm, at most 1.95 mm, at most 2 mm, at most 2.05 mm, at most 2.1 mm, at most 2.15 mm, at most 2.2 mm, at most 2.25 mm, at most 2.3 mm, at most 2.35 mm, at most 2.4 mm, at most 2.45 mm, at most 2.5 mm, at most 2.55 mm, at most 2.6 mm, at most 2.65 mm, at most 2.7 mm, at most 2.75 mm, at most 2.8 mm, at most 2.85 mm, at most 2.9 mm, at most 2.95 mm, at most 3 mm, at most 3.05 mm, at most 3.1 mm, at most 3.15 mm, at most 3.2 mm, at most 3.25 mm, at most 3.3 mm, at most 3.35 mm, at most 3.4 mm, at most 3.45 mm, at most 3.5 mm, at most 3.55 mm, at most 3.6 mm, at most 3.65 mm, at most 3.7 mm, at most 3.75 mm, at most 3.8 mm, at most 3.85 mm, at most 3.9 mm, or at most 3.95 mm. In some embodiments, the maximum diameter of the bulb 101 is the same as the maximum diameter of the handle 106. In some embodiments, the maximum diameter of the bulb 101 is less than the maximum diameter of the handle 106. In some embodiments, the maximum diameter of the bulb 101 is greater than the maximum diameter of the handle 106.

In some embodiments, the handle 106 comprises a flattened portion 107 (see e.g., FIG. 6). In some embodiments, the handle 106 is combination of a cylindrical rod (e.g., closer to the head 102) and the flattened portion 107 (e.g., closer to the proximal end of the swab 100). In some embodiments, the handle 106 is combination of a rod comprising a polygonal cross-section (e.g., closer to the head 102) and flattened portion 107 (e.g., closer to the proximal end of the swab 100). In some embodiments, the flattened portion 107 of the handle 106 comprises a raised edge. In some embodiments, the flattened portion 107 of the handle 106 comprises a location for insertion of a barcode or label. In some embodiments, the flattened portion 107 of the handle 106 can comprise any shape or combination thereof, e.g., quadrilateral, rounded quadrilateral, circular, oval, triangular, etc. In some embodiments, the flattened portion 107 of the handle 106 comprises a rounded quadrilateral (e.g., at the proximal end of the handle 106) connected to a second quadrilateral with a shorter width than the first quadrilateral (e.g., at the distal end of the handle 106).

In some embodiments, the flattened portion 107 of the handle 106 has a maximum width (e.g., when viewing the flattened portion 107 of the handle 106 straight-on; e.g., the maximum width of the larger rounded quadrilateral) of about 8.5 mm. In some embodiments, the flattened portion 107 of the handle 106 has a maximum width of about 8.58 mm. In some embodiments, the flattened portion 107 of the handle 106 has a maximum width of about 5 mm-10 mm. In some embodiments, the flattened portion 107 of the handle 106 has a maximum width of at least 5 mm, at least 5.1 mm, at least 5.2 mm, at least 5.3 mm, at least 5.4 mm, at least 5.5 mm, at least 5.6 mm, at least 5.7 mm, at least 5.8 mm, at least 5.9 mm, at least 6 mm, at least 6.1 mm, at least 6.2 mm, at least 6.3 mm, at least 6.4 mm, at least 6.5 mm, at least 6.6 mm, at least 6.7 mm, at least 6.8 mm, at least 6.9 mm, at least 7 mm, at least 7.1 mm, at least 7.2 mm, at least 7.3 mm, at least 7.4 mm, at least 7.5 mm, at least 7.6 mm, at least 7.7 mm, at least 7.8 mm, at least 7.9 mm, at least 8 mm, at least 8.1 mm, at least 8.2 mm, at least 8.3 mm, at least 8.4 mm, at least 8.5 mm, at least 8.6 mm, at least 8.7 mm, at least 8.8 mm, at least 8.9 mm, at least 9 mm, at least 9.1 mm, at least 9.2 mm, at least 9.3 mm, at least 9.4 mm, at least 9.5 mm, at least 9.6 mm, at least 9.7 mm, at least 9.8 mm, at least 9.9 mm, or at least 10 mm.

In some embodiments, the flattened portion 107 of the handle 106 has a maximum width of at most 5 mm, at most 5.1 mm, at most 5.2 mm, at most 5.3 mm, at most 5.4 mm, at most 5.5 mm, at most 5.6 mm, at most 5.7 mm, at most 5.8 mm, at most 5.9 mm, at most 6 mm, at most 6.1 mm, at most 6.2 mm, at most 6.3 mm, at most 6.4 mm, at most 6.5 mm, at most 6.6 mm, at most 6.7 mm, at most 6.8 mm, at most 6.9 mm, at most 7 mm, at most 7.1 mm, at most 7.2 mm, at most 7.3 mm, at most 7.4 mm, at most 7.5 mm, at most 7.6 mm, at most 7.7 mm, at most 7.8 mm, at most 7.9 mm, at most 8 mm, at most 8.1 mm, at most 8.2 mm, at most 8.3 mm, at most 8.4 mm, at most 8.5 mm, at most 8.6 mm, at most 8.7 mm, at most 8.8 mm, at most 8.9 mm, at most 9 mm, at most 9.1 mm, at most 9.2 mm, at most 9.3 mm, at most 9.4 mm, at most 9.5 mm, at most 9.6 mm, at most 9.7 mm, at most 9.8 mm, at most 9.9 mm, or at most 10 mm.

In some embodiments, the flattened portion 107 of the handle 106 has a minimum width (e.g., when viewing the flattened portion 107 of the handle 106 straight-on; e.g., the width of the smaller rounded quadrilateral) of about 2 mm. In some embodiments, the flattened portion 107 of the handle 106 has a minimum width of about 1.5 mm-5 mm. In some embodiments, the flattened portion 107 of the handle 106 has a minimum width of at least 1.5 mm, at least 1.6 mm, at least 1.7 mm, at least 1.8 mm, at least 1.9 mm, at least 2 mm, at least 2.1 mm, at least 2.2 mm, at least 2.3 mm, at least 2.4 mm, at least 2.5 mm, at least 2.6 mm, at least 2.7 mm, at least 2.8 mm, at least 2.9 mm, at least 3 mm, at least 3.1 mm, at least 3.2 mm, at least 3.3 mm, at least 3.4 mm, at least 3.5 mm, at least 3.6 mm, at least 3.7 mm, at least 3.8 mm, at least 3.9 mm, at least 4 mm, at least 4.1 mm, at least 4.2 mm, at least 4.3 mm, at least 4.4 mm, at least 4.5 mm, at least 4.6 mm, at least 4.7 mm, at least 4.8 mm, at least 4.9 mm, or at least 5 mm. In some embodiments, the flattened portion 107 of the handle 106 has a minimum width of at most 1.5 mm, at most 1.6 mm, at most 1.7 mm, at most 1.8 mm, at most 1.9 mm, at most 2 mm, at most 2.1 mm, at most 2.2 mm, at most 2.3 mm, at most 2.4 mm, at most 2.5 mm, at most 2.6 mm, at most 2.7 mm, at most 2.8 mm, at most 2.9 mm, at most 3 mm, at most 3.1 mm, at most 3.2 mm, at most 3.3 mm, at most 3.4 mm, at most 3.5 mm, at most 3.6 mm, at most 3.7 mm, at most 3.8 mm, at most 3.9 mm, at most 4 mm, at most 4.1 mm, at most 4.2 mm, at most 4.3 mm, at most 4.4 mm, at most 4.5 mm, at most 4.6 mm, at most 4.7 mm, at most 4.8 mm, at most 4.9 mm, or at most 5 mm.

In some embodiments, the flattened portion 107 of the handle 106 has a thickness (e.g., when viewing the flattened portion 107 of the handle 106 edge-on) of about 2 mm. In some embodiments, the flattened portion 107 of the handle 106 has a thickness of about 1 mm-5 mm. In some embodiments, the flattened portion 107 of the handle 106 has a thickness of at least 1 mm, at least 1.1 mm, at least 1.2 mm, at least 1.3 mm, at least 1.4 mm, at least 1.5 mm, at least 1.6 mm, at least 1.7 mm, at least 1.8 mm, at least 1.9 mm, at least 2 mm, at least 2.1 mm, at least 2.2 mm, at least 2.3 mm, at least 2.4 mm, at least 2.5 mm, at least 2.6 mm, at least 2.7 mm, at least 2.8 mm, at least 2.9 mm, at least 3 mm, at least 3.1 mm, at least 3.2 mm, at least 3.3 mm, at least 3.4 mm, at least 3.5 mm, at least 3.6 mm, at least 3.7 mm, at least 3.8 mm, at least 3.9 mm, at least 4 mm, at least 4.1 mm, at least 4.2 mm, at least 4.3 mm, at least 4.4 mm, at least 4.5 mm, at least 4.6 mm, at least 4.7 mm, at least 4.8 mm, at least 4.9 mm, or at least 5 mm.

In some embodiments, the flattened portion 107 of the handle 106 has a thickness of at most 1 mm, at most 1.1 mm, at most 1.2 mm, at most 1.3 mm, at most 1.4 mm, at most 1.5 mm, at most 1.6 mm, at most 1.7 mm, at most 1.8 mm, at most 1.9 mm, at most 2 mm, at most 2.1 mm, at most 2.2 mm, at most 2.3 mm, at most 2.4 mm, at most 2.5 mm, at most 2.6 mm, at most 2.7 mm, at most 2.8 mm, at most 2.9 mm, at most 3 mm, at most 3.1 mm, at most 3.2 mm, at most 3.3 mm, at most 3.4 mm, at most 3.5 mm, at most 3.6 mm, at most 3.7 mm, at most 3.8 mm, at most 3.9 mm, at most 4 mm, at most 4.1 mm, at most 4.2 mm, at most 4.3 mm, at most 4.4 mm, at most 4.5 mm, at most 4.6 mm, at most 4.7 mm, at most 4.8 mm, at most 4.9 mm, or at most 5 mm.

In some embodiments, the flattened portion 107 of the handle 106 has a length (e.g., from the proximal to distal end of the handle 106) of about 26 mm. In some embodiments, the flattened portion 107 of the handle 106 has a length of about 10 mm-40 mm. In some embodiments, the flattened portion 107 of the handle 106 has a length of at least 10 mm, at least 11 mm, at least 12 mm, at least 13 mm, at least 14 mm, at least 15 mm, at least 16 mm, at least 17 mm, at least 18 mm, at least 19 mm, at least 20 mm, at least 21 mm, at least 22 mm, at least 23 mm, at least 24 mm, at least 25 mm, at least 26 mm, at least 27 mm, at least 28 mm, at least 29 mm, at least 30 mm, at least 31 mm, at least 32 mm, at least 33 mm, at least 34 mm, at least 35 mm, at least 36 mm, at least 37 mm, at least 38 mm, at least 39 mm, or at least 40 mm. In some embodiments, the flattened portion 107 of the handle 106 has a length of at most 10 mm, at most 11 mm, at most 12 mm, at most 13 mm, at most 14 mm, at most 15 mm, at most 16 mm, at most 17 mm, at most 18 mm, at most 19 mm, at most 20 mm, at most 21 mm, at most 22 mm, at most 23 mm, at most 24 mm, at most 25 mm, at most 26 mm, at most 27 mm, at most 28 mm, at most 29 mm, at most 30 mm, at most 31 mm, at most 32 mm, at most 33 mm, at most 34 mm, at most 35 mm, at most 36 mm, at most 37 mm, at most 38 mm, at most 39 mm, or at most 40 mm.

### Breakpoint

In some embodiments, the swab 100 further comprises a breakpoint 108. As used herein, the term “breakpoint” refers to a portion of the swab 100 with a minimal diameter such that application of force separates the swab 100 into two pieces at the breakpoint 108. In some embodiments, a break at the breakpoint 108 can be accomplished by a single direction bend. In some embodiments, a break at the breakpoint 108 can be accomplished by torsion (e.g., twisting). In some embodiments, a break at the breakpoint 108 can be accomplished by a single direction bend combined with torsion, before and/or after the bend or at the same time as the bend. As a non-limiting example, a swab material such as polypropylene can require a torsion in order to break the swab 100 at the breakpoint 108. In some embodiments, need for torsion to break the swab 100 at the breakpoint 108 can make the swab 100 less prone to accidental breakage. In some embodiments, the swab 100 comprises one breakpoint 108. In some embodiments, the swab 100 comprises 2, 3, 4, 5 or more breakpoint 108s. In some embodiments, the breakpoint 108 is proximal to the head 102. In some embodiments, the breakpoint 108 is located between the neck 104 and the handle 106. In some embodiments, the breakpoint 108 is located in the neck 104. In some embodiments, the breakpoint 108 is located in the handle 106. In some embodiments, the cross-section of the breakpoint 108 is a circle, a semicircle, a truncated circle, or a circle with one or more flat sides. In some embodiments, the cross-section of the breakpoint 108 is a circle. In some embodiments, the breakpoint 108 has a polygonal cross section, e.g., a cross-section in the shape of a triangle, a square, a quadrilateral, a trapezoid, a pentagon, a hexagon, or a polygon with at least 7, 8, 9, 10, 11, 12, 13, 14, 15, 16, 17, 18, 19, 20 or more sides. In some embodiments, at least one side of the cross-section of the breakpoint 108 comprises a convex and/or concave curve. In some embodiments, the cross section of the breakpoint 108 is a rotationally symmetric shape. In some embodiments, the cross section of the breakpoint 108 is an asymmetric shape. In some embodiments, the breakpoint 108 cross-section is the same for the entirety of the breakpoint 108. In some embodiments, the breakpoint 108 cross-section is different for at least one portion of the breakpoint 108; the breakpoint 108 can comprise any combination of different (e.g., at least 2, at least 3, at least 4, at least 5) breakpoint cross-sections.

In some embodiments, the breakpoint 108 has a minimum diameter that is less than the minimum diameter of the handle 106. In some embodiments, the breakpoint 108 has a minimum diameter that is less than the maximum diameter of the neck 104. In some embodiments, the breakpoint 108 tapers from a first maximum diameter (e.g., closer to the handle 106 end) to a minimum dimeter (e.g., the breakpoint 108), and tapers from the minimum diameter to a second maximum diameter. In some embodiments, the first maximum diameter of the breakpoint 108 is the same as the minimum diameter of the handle 106. In some embodiments, the first maximum diameter of the breakpoint 108 is less than the minimum diameter of the handle 106. In some embodiments, the first maximum diameter of the breakpoint 108 is greater than the minimum diameter of the handle 106. In some embodiments, the first maximum diameter of the breakpoint 108 is the same as the maximum diameter of the handle 106. In some embodiments, the first maximum diameter of the breakpoint 108 is less than the maximum diameter of the handle 106. In some embodiments, the second maximum diameter of the breakpoint 108 is the same as the maximum diameter of the neck 104. In some embodiments, the second maximum diameter of the breakpoint 108 is less than the maximum diameter of the neck 104. In some embodiments, the second maximum diameter of the breakpoint 108 is greater than the maximum diameter of the neck 104. In some embodiments, the tapering of the breakpoint 108 is linear. In some embodiments, the tapering of the breakpoint 108 is rounded. In some embodiments, the tapering of the breakpoint 108 is concentric (e.g., aligns with the central axis of the swab 100). In some embodiments, the tapering of the breakpoint 108 is biased to one side (e.g., does not align with the central axis of the swab 100).

In some embodiments, the breakpoint 108 has a minimum diameter of about 0.5 mm. In some embodiments, the breakpoint 108 has a minimum diameter of about 0.1 mm-1 mm. In some embodiments, the breakpoint 108 has a minimum diameter of at least 0.1 mm, at least 0.15 mm, at least 0.2 mm, at least 0.25 mm, at least 0.3 mm, at least 0.35 mm, at least 0.4 mm, at least 0.45 mm, at least 0.5 mm, at least 0.55 mm, at least 0.6 mm, at least 0.65 mm, at least 0.7 mm, at least 0.75 mm, at least 0.8 mm, at least 0.85 mm, at least 0.9 mm, at least 0.95 mm, or at least 1.0 mm. In some embodiments, the breakpoint 108 has a minimum diameter of at most 0.1 mm, at most 0.15 mm, at most 0.2 mm, at most 0.25 mm, at most 0.3 mm, at most 0.35 mm, at most 0.4 mm, at most 0.45 mm, at most 0.5 mm, at most 0.55 mm, at most 0.6 mm, at most 0.65 mm, at most 0.7 mm, at most 0.75 mm, at most 0.8 mm, at most 0.85 mm, at most 0.9 mm, at most 0.95 mm, or at most 1.0 mm.

In some embodiments, the breakpoint 108 has a maximum (e.g., first or second maximum) diameter of about 1.5 mm. In some embodiments, the breakpoint 108 has a maximum (e.g., first or second maximum) diameter of about 1.0 mm-10.0 mm. In some embodiments, the breakpoint 108 has a maximum (e.g., first or second maximum) diameter of at least 1 mm, at least 1.05 mm, at least 1.1 mm, at least 1.15 mm, at least 1.2 mm, at least 1.25 mm, at least 1.3 mm, at least 1.35 mm, at least 1.4 mm, at least 1.45 mm, at least 1.5 mm, at least 1.55 mm, at least 1.6 mm, at least 1.65 mm, at least 1.7 mm, at least 1.75 mm, at least 1.8 mm, at least 1.85 mm, at least 1.9 mm, at least 1.95 mm, at least 2 mm, at least 2.05 mm, at least 2.1 mm, at least 2.15 mm, at least 2.2 mm, at least 2.25 mm, at least 2.3 mm, at least 2.35 mm, at least 2.4 mm, at least 2.45 mm, at least 2.5 mm, at least 2.55 mm, at least 2.6 mm, at least 2.65 mm, at least 2.7 mm, at least 2.75 mm, at least 2.8 mm, at least 2.85 mm, at least 2.9 mm, at least 2.95 mm, at least 3 mm, at least 3.05 mm, at least 3.1 mm, at least 3.15 mm, at least 3.2 mm, at least 3.25 mm, at least 3.3 mm, at least 3.35 mm, at least 3.4 mm, at least 3.45 mm, at least 3.5 mm, at least 3.55 mm, at least 3.6 mm, at least 3.65 mm, at least 3.7 mm, at least 3.75 mm, at least 3.8 mm, at least 3.85 mm, at least 3.9 mm, at least 3.95 mm, at least 4.0 mm, at least 4.5 mm, at least 5 mm, at least 5.5 mm, at least 6 mm, at least 6.5 mm, at least 7 mm, at least 7.5 mm, at least 8 mm, at least 8.5 mm, at least 9 mm, at least 9.5 mm, or at least 10 mm.

In some embodiments, the breakpoint 108 has a maximum (e.g., first or second maximum) diameter of at most 1 mm, at most 1.05 mm, at most 1.1 mm, at most 1.15 mm, at most 1.2 mm, at most 1.25 mm, at most 1.3 mm, at most 1.35 mm, at most 1.4 mm, at most 1.45 mm, at most 1.5 mm, at most 1.55 mm, at most 1.6 mm, at most 1.65 mm, at most 1.7 mm, at most 1.75 mm, at most 1.8 mm, at most 1.85 mm, at most 1.9 mm, at most 1.95 mm, at most 2 mm, at most 2.05 mm, at most 2.1 mm, at most 2.15 mm, at most 2.2 mm, at most 2.25 mm, at most 2.3 mm, at most 2.35 mm, at most 2.4 mm, at most 2.45 mm, at most 2.5 mm, at most 2.55 mm, at most 2.6 mm, at most 2.65 mm, at most 2.7 mm, at most 2.75 mm, at most 2.8 mm, at most 2.85 mm, at most 2.9 mm, at most 2.95 mm, at most 3 mm, at most 3.05 mm, at most 3.1 mm, at most 3.15 mm, at most 3.2 mm, at most 3.25 mm, at most 3.3 mm, at most 3.35 mm, at most 3.4 mm, at most 3.45 mm, at most 3.5 mm, at most 3.55 mm, at most 3.6 mm, at most 3.65 mm, at most 3.7 mm, at most 3.75 mm, at most 3.8 mm, at most 3.85 mm, at most 3.9 mm, at most 3.95 mm, at most 4.0 mm, at most 4.5 mm, at most 5 mm, at most 5.5 mm, at most 6 mm, at most 6.5 mm, at most 7 mm, at most 7.5 mm, at most 8 mm, at most 8.5 mm, at most 9 mm, at most 9.5 mm, or at most 10 mm.

### Plunger

In some embodiments, the swab 100 further comprises a plunger 110. As used herein, the term “plunger 110” refers to a short portion of the swab 100 with a large diameter or width, e.g., which can function as a cap or barrier within a container tube 112 or protects the user’s hands from the sample (see e.g., FIGS. 5-6). In some embodiments, the plunger 110 has a maximum diameter greater than the minimum diameter of the anterior nare or nasal cavity, such that the swab 100 is only inserted up to plunger 110. In some embodiments, the shape of the plunger 110 is a truncated cone. In some embodiments, the shape of the plunger 110 is a truncated prism. In some embodiments, the cross-sectional shape of the plunger 110 is the same as that of the container tube 112. In some embodiments, the cross-section of the plunger 110 is a circle, a semicircle, a truncated circle, or a circle with one or more flat sides. In some embodiments, the cross-section of the plunger 110 is a circle. In some embodiments, the plunger 110 comprises a polygonal cross section, e.g., a cross-section in the shape of a triangle, a square, a quadrilateral, a trapezoid, a pentagon, a hexagon, or a polygon with at least 7, 8, 9, 10, 11, 12, 13, 14, 15, 16, 17, 18, 19, 20 or more sides. In some embodiments, at least one side of the cross-section of the plunger 110 comprises a convex and/or concave curve. In some embodiments, the cross section of the plunger 110 is a rotationally symmetric shape. In some embodiments, the cross section of the plunger 110 is an asymmetric shape. In some embodiments, the plunger cross-section is the same for the entirety of the plunger 110. In some embodiments, the plunger cross-section is different for at least one portion of the plunger 110; the plunger 110 can comprise any combination of different (e.g., at least 2, at least 3, at least 4, at least 5) plunger cross-sections. In some embodiments, the plunger 110 is located between the neck 104 and handle 106. In some embodiments, the plunger 110 is connected to the neck 104 and handle 106. In some embodiments, the plunger 110 is located between the neck 104 and a flattened portion 107 of the handle 106. Accordingly, in one aspect, the swab 100 comprises: (a) the collection head 102, (b) the neck 104, (c) the plunger 110, and (d) the handle 106 with the flattened portion 107.

In some embodiments, the plunger 110 tapers from a maximum diameter (e.g., closer to the handle 106) to a minimum dimeter (e.g., closer to the neck 104). In some embodiments, the maximum diameter of the plunger 110 is greater than the maximum diameter or width of the handle 106. In some embodiments, the maximum diameter of the plunger 110 is the same as the maximum diameter or width of the handle 106. In some embodiments, the maximum diameter of the plunger 110 is less than the maximum diameter or width of the handle 106. In some embodiments, the maximum diameter of the plunger 110 is the same as the internal diameter of the container tube 112. In some embodiments, the minimum diameter of the plunger 110 is the same as the maximum diameter of the neck 104. In some embodiments, the minimum diameter of the plunger 110 is greater than the maximum diameter of the neck 104. In some embodiments, the minimum diameter of the plunger 110 is less than the maximum diameter of the neck 104. In some embodiments, the tapering of the plunger 110 is linear. In some embodiments, the tapering of the plunger 110 is rounded.

In some embodiments, the plunger 110 has a maximum diameter of about 10 mm. In some embodiments, the plunger 110 has a maximum diameter of about 4 mm-20 mm. In some embodiments, the plunger 110 has a maximum diameter that does not permit entry into the nasal cavity. In some embodiments, the plunger 110 has a maximum diameter of at least 4 mm. In some embodiments, the plunger 110 has a maximum diameter of at least 4 mm, at least 4.5 mm, at least 5 mm, at least 5.5 mm, at least 6 mm, at least 6.5 mm, at least 7 mm, at least 7.5 mm, at least 8 mm, at least 8.5 mm, at least 9 mm, at least 9.5 mm, at least 10 mm, at least 10.5 mm, at least 11 mm, at least 11.5 mm, at least 12 mm, at least 12.5 mm, at least 13 mm, at least 13.5 mm, at least 14 mm, at least 14.5 mm, at least 15 mm, at least 15.5 mm, at least 16 mm, at least 16.5 mm, at least 17 mm, at least 17.5 mm, at least 18 mm, at least 18.5 mm, at least 19 mm, at least 19.5 mm, or at least 20 mm. In some embodiments, the plunger 110 has a maximum diameter of about 4 mm-20 mm. at most 4 mm, at most 4.5 mm, at most 5 mm, at most 5.5 mm, at most 6 mm, at most 6.5 mm, at most 7 mm, at most 7.5 mm, at most 8 mm, at most 8.5 mm, at most 9 mm, at most 9.5 mm, at most 10 mm, at most 10.5 mm, at most 11 mm, at most 11.5 mm, at most 12 mm, at most 12.5 mm, at most 13 mm, at most 13.5 mm, at most 14 mm, at most 14.5 mm, at most 15 mm, at most 15.5 mm, at most 16 mm, at most 16.5 mm, at most 17 mm, at most 17.5 mm, at most 18 mm, at most 18.5 mm, at most 19 mm, at most 19.5 mm, or at most 20 mm.

In some embodiments, the plunger 110 has a minimum diameter (e.g., towards the neck 104 or head 102) of about 1.0 mm-4.0 mm. In some embodiments, the plunger 110 has a minimum diameter of about 1.5 mm. In some embodiments, the plunger 110 has a minimum diameter of at least 1 mm, at least 1.05 mm, at least 1.1 mm, at least 1.15 mm, at least 1.2 mm, at least 1.25 mm, at least 1.3 mm, at least 1.35 mm, at least 1.4 mm, at least 1.45 mm, at least 1.5 mm, at least 1.55 mm, at least 1.6 mm, at least 1.65 mm, at least 1.7 mm, at least 1.75 mm, at least 1.8 mm, at least 1.85 mm, at least 1.9 mm, at least 1.95 mm, at least 2 mm, at least 2.05 mm, at least 2.1 mm, at least 2.15 mm, at least 2.2 mm, at least 2.25 mm, at least 2.3 mm, at least 2.35 mm, at least 2.4 mm, at least 2.45 mm, at least 2.5 mm, at least 2.55 mm, at least 2.6 mm, at least 2.65 mm, at least 2.7 mm, at least 2.75 mm, at least 2.8 mm, at least 2.85 mm, at least 2.9 mm, at least 2.95 mm, at least 3 mm, at least 3.05 mm, at least 3.1 mm, at least 3.15 mm, at least 3.2 mm, at least 3.25 mm, at least 3.3 mm, at least 3.35 mm, at least 3.4 mm, at least 3.45 mm, at least 3.5 mm, at least 3.55 mm, at least 3.6 mm, at least 3.65 mm, at least 3.7 mm, at least 3.75 mm, at least 3.8 mm, at least 3.85 mm, at least 3.9 mm, at least 3.95 mm, or at least 4.0 mm.

In some embodiments, the plunger 110 has a minimum diameter of at most 1 mm, at most 1.05 mm, at most 1.1 mm, at most 1.15 mm, at most 1.2 mm, at most 1.25 mm, at most 1.3 mm, at most 1.35 mm, at most 1.4 mm, at most 1.45 mm, at most 1.5 mm, at most 1.55 mm, at most 1.6 mm, at most 1.65 mm, at most 1.7 mm, at most 1.75 mm, at most 1.8 mm, at most 1.85 mm, at most 1.9 mm, at most 1.95 mm, at most 2 mm, at most 2.05 mm, at most 2.1 mm, at most 2.15 mm, at most 2.2 mm, at most 2.25 mm, at most 2.3 mm, at most 2.35 mm, at most 2.4 mm, at most 2.45 mm, at most 2.5 mm, at most 2.55 mm, at most 2.6 mm, at most 2.65 mm, at most 2.7 mm, at most 2.75 mm, at most 2.8 mm, at most 2.85 mm, at most 2.9 mm, at most 2.95 mm, at most 3 mm, at most 3.05 mm, at most 3.1 mm, at most 3.15 mm, at most 3.2 mm, at most 3.25 mm, at most 3.3 mm, at most 3.35 mm, at most 3.4 mm, at most 3.45 mm, at most 3.5 mm, at most 3.55 mm, at most 3.6 mm, at most 3.65 mm, at most 3.7 mm, at most 3.75 mm, at most 3.8 mm, at most 3.85 mm, at most 3.9 mm, at most 3.95 mm, or at most 4.0 mm.

In some embodiments, the plunger 110 has a thickness (e.g., from the proximal to the distal end of the plunger 110) of about 1 mm-10 mm. In some embodiments, the plunger 110 has a thickness of about 5 mm. In some embodiments, the plunger 110 has a thickness of at least 1 mm, at least 1.5 mm, at least 2 mm, at least 2.5 mm, at least 3 mm, at least 3.5 mm, at least 4 mm, at least 4.5 mm, at least 5 mm, at least 5.5 mm, at least 6 mm, at least 6.5 mm, at least 7 mm, at least 7.5 mm, at least 8 mm, at least 8.5 mm, at least 9 mm, at least 9.5 mm, or at least 10 mm. In some embodiments, the plunger 110 has a thickness of at most 1 mm, at most 1.5 mm, at most 2 mm, at most 2.5 mm, at most 3 mm, at most 3.5 mm, at most 4 mm, at most 4.5 mm, at most 5 mm, at most 5.5 mm, at most 6 mm, at most 6.5 mm, at most 7 mm, at most 7.5 mm, at most 8 mm, at most 8.5 mm, at most 9 mm, at most 9.5 mm, or at most 10 mm.

### Materials

In some embodiments, the swab material exhibits at least one of the following characteristics: (1) It is sufficiently rigid for collection of cells (e.g., from the back of the throat). (2) It is sufficiently flexible for safety of use. (3) It collects adequate sample from the patient for subsequent tests (e.g., for viral infection). (4) It withstands the rigors of sterilization/disinfection without a) structural weakening, or b) chemically interfering with PCR testing. (5) It is compatible with standard PCR testing. In some embodiments, the swab material is biodegradable and/or water-soluble.

In some embodiments, the swab 100 is constructed from a semi-flexible material, such as polypropylene, polycarbonate, thermoplastic elastomers (TPE), rubber, polyester fiber, acrylonitrile butadiene styrene (ABS), acrylic, polyetherimide, ionomer, acetal copolymer, polyurethane, polystyrene, nylon, and the like, or any combination thereof. In some embodiments, the swab material is a flexible polymer. In some embodiments, the swab material is a solid material (e.g., non-porous). In some embodiments, the swab material is a foam. In some embodiments, the swab material is a porous material. In some embodiments, all of the components of the swab 100 (e.g., head 102, neck 104, handle 106, breakpoint 108, and/or plunger 110) comprise the same material. In some embodiments, at least one component of the swab 100 (e.g., head 102, neck 104, handle 106, breakpoint 108, and/or plunger 110) is made from a different material from the remainder of the swab 100. In some embodiments, the swab 100 comprises at least 2 (e.g., 2, 3, 4, 5, or more) materials as described herein. As a non-limiting example, a swab 100 comprising at least two materials can be accomplished using injection molding (e.g., overmolding). Overmolding is a process wherein a single part is created using two or more different materials in combination. Typically, the first material, sometimes referred to as the substrate, is partially or fully covered by subsequent materials (e.g., overmold materials) during the manufacturing process.

In some embodiments, the swab material comprises polypropylene. In some embodiments, the polypropylene swab material comprises Flint Hills Resources™ (FHR) PSM4R polypropylene copolymer. In some embodiments, the polypropylene swab material comprises a random copolymer for injection molding. In some embodiments, the swab material exhibits the following features: autoclave sterilizable; E-beam sterilizable; ethylene oxide sterilizable; no animal derived components; and radiation sterilizable.

In some embodiments, the swab material does not comprise nylon. In some embodiments, the swab material does not comprise polystyrene. In some embodiments, the swab material is hydrophobic. In some embodiments, at least one component of the swab 100 is a different material than other components of the swab 100. In some embodiments, the swab 100 comprises 1, 2, 3, 4, 5, or more different materials.

In some embodiments, the swab material has a flexural modulus of about 500 megapascals (MPa) to 800 MPa. As used herein, the term “flexural modulus” (also referred to as bending modulus) is the ratio of stress to strain in flexural deformation, or the tendency for a material to resist bending. In some embodiments, the swab material has a tangent flexural modulus of about 790 MPa. In some embodiments, the swab material has a flexural modulus of about 500 MPa to 2000 MPa. In some embodiments, the swab material has a flexural modulus of about 100 MPa to 5000 MPa.

In some embodiments, the swab material has a flexural modulus of at least 100 MPa, at least 150 MPa, at least 200 MPa, at least 250 MPa, at least 300 MPa, at least 350 MPa, at least 400 MPa, at least 450 MPa, at least 500 MPa, at least 500 MPa, at least 510 MPa, at least 520 MPa, at least 530 MPa, at least 540 MPa, at least 550 MPa, at least 560 MPa, at least 570 MPa, at least 580 MPa, at least 590 MPa, at least 600 MPa, at least 610 MPa, at least 620 MPa, at least 630 MPa, at least 640 MPa, at least 650 MPa, at least 660 MPa, at least 670 MPa, at least 680 MPa, at least 690 MPa, at least 700 MPa, at least 710 MPa, at least 720 MPa, at least 730 MPa, at least 740 MPa, at least 750 MPa, at least 760 MPa, at least 770 MPa, at least 780 MPa, at least 790 MPa, at least 800 MPa, at least 850 MPa, at least 900 MPa, at least 950 MPa, at least 1000 MPa, at least 1050 MPa, at least 1100 MPa, at least 1150 MPa, at least 1200 MPa, at least 1250 MPa, at least 1300 MPa, at least 1350 MPa, at least 1400 MPa, at least 1450 MPa, at least 1500 MPa, at least 1550 MPa, at least 1600 MPa, at least 1650 MPa, at least 1700 MPa, at least 1750 MPa, at least 1800 MPa, at least 1850 MPa, at least 1900 MPa, at least 1950 MPa, at least 2000 MPa, at least 2000 MPa, at least 2100 MPa, at least 2200 MPa, at least 2300 MPa, at least 2400 MPa, at least 2500 MPa, at least 2600 MPa, at least 2700 MPa, at least 2800 MPa, at least 2900 MPa, at least 3000 MPa, at least 3100 MPa, at least 3200 MPa, at least 3300 MPa, at least 3400 MPa, at least 3500 MPa, at least 3600 MPa, at least 3700 MPa, at least 3800 MPa, at least 3900 MPa, at least 4000 MPa, at least 4100 MPa, at least 4200 MPa, at least 4300 MPa, at least 4400 MPa, at least 4500 MPa, at least 4600 MPa, at least 4700 MPa, at least 4800 MPa, at least 4900 MPa, or at least 5000 MPa.

In some embodiments, the swab material has a flexural modulus of at most 100 MPa, at most 150 MPa, at most 200 MPa, at most 250 MPa, at most 300 MPa, at most 350 MPa, at most 400 MPa, at most 450 MPa, at most 500 MPa, at most 500 MPa, at most 510 MPa, at most 520 MPa, at most 530 MPa, at most 540 MPa, at most 550 MPa, at most 560 MPa, at most 570 MPa, at most 580 MPa, at most 590 MPa, at most 600 MPa, at most 610 MPa, at most 620 MPa, at most 630 MPa, at most 640 MPa, at most 650 MPa, at most 660 MPa, at most 670 MPa, at most 680 MPa, at most 690 MPa, at most 700 MPa, at most 710 MPa, at most 720 MPa, at most 730 MPa, at most 740 MPa, at most 750 MPa, at most 760 MPa, at most 770 MPa, at most 780 MPa, at most 790 MPa, at most 800 MPa, at most 850 MPa, at most 900 MPa, at most 950 MPa, at most 1000 MPa, at most 1050 MPa, at most 1100 MPa, at most 1150 MPa, at most 1200 MPa, at most 1250 MPa, at most 1300 MPa, at most 1350 MPa, at most 1400 MPa, at most 1450 MPa, at most 1500 MPa, at most 1550 MPa, at most 1600 MPa, at most 1650 MPa, at most 1700 MPa, at most 1750 MPa, at most 1800 MPa, at most 1850 MPa, at most 1900 MPa, at most 1950 MPa, at most 2000 MPa, at most 2000 MPa, at most 2100 MPa, at most 2200 MPa, at most 2300 MPa, at most 2400 MPa, at most 2500 MPa, at most 2600 MPa, at most 2700 MPa, at most 2800 MPa, at most 2900 MPa, at most 3000 MPa, at most 3100 MPa, at most 3200 MPa, at most 3300 MPa, at most 3400 MPa, at most 3500 MPa, at most 3600 MPa, at most 3700 MPa, at most 3800 MPa, at most 3900 MPa, at most 4000 MPa, at most 4100 MPa, at most 4200 MPa, at most 4300 MPa, at most 4400 MPa, at most 4500 MPa, at most 4600 MPa, at most 4700 MPa, at most 4800 MPa, at most 4900 MPa, or at most 5000 MPa.

In another aspect, described herein is a swab 100 constructed from a water-soluble or biodegradable material. In some embodiments, the swab material is biodegradable and water-soluble. In some embodiments, the swab material is biodegradable. In some embodiments, the swab material is water-soluble. In some embodiments, the swab material is a foam. In some embodiments, the swab material is a porous material. Non-limiting examples of biodegradable swab materials include a bio-based plastic, polyhydroxyalkanoate (PHA), polylactic acid (PLA), starch blend, cellulose-based plastic, lignin-based polymer composite, a petroleum-based plastic, polyglycolic acid (PGA), polybutylene succinate (PBS), polycaprolactone (PCL), poly(vinyl alcohol) (PVA, PVOH), or polybutylene adipate terephthalate (PBAT). In some embodiments the swab material comprises polyvinyl alcohol or a derivative polymer such as polyvinyl acetals, polyvinyl butyral (PVB), or polyvinyl formal (PVF). In some embodiments, the swab material comprises Kuraray MOWIFLEX™ C17 or C30 materials, which are PVA variants. In some embodiments, the material consists essentially of polyvinyl alcohol. In some embodiments, the material (e.g., polyvinyl alcohol) does not interfere with downstream applications (e.g., PCR, qPCR, RT-qPCR, isothermal amplification, RPA, etc.). In some embodiments, the sample collection head 102 comprises a first material, and the remainder of the swab 100 (e.g. handle 106, neck 104, breakpoint 108, and/or plunger 110) comprises a second material. As a non-limiting example, the sample collection head 102 comprises a water-soluble and/or biodegradable material and the remainder of the swab 100 comprises a flexible polymer. As a non-limiting example, the sample collection head 102 comprises PVA and the remainder of the swab 100 comprises polypropylene.

### Kits

Another aspect of the technology described herein relates to kits for collecting samples using the swabs 100 as described herein. Described herein are kit components that can be included in one or more of the kits described herein.

In some embodiments, the kit comprises a swab 100 as described herein. In some embodiments, the kit comprises a swab 100 comprising a sample collection head 102, a neck 104, and a handle 106. In some embodiments, the kit comprises a swab 100 comprising a sample collection head 102, a neck 104, a breakpoint 108, and a handle 106. In some embodiments, the kit comprises a swab 100 comprising a sample collection head 102, a neck 104, a plunger 110, and a (e.g., flattened) handle 106. In some embodiments, the kit comprises 1, 2, 3, 4, 5, 6, 7, 8, 9, 10, 11, 12, 13, 14, 15, 16, 17, 18, 19, 20 or more swabs 100 as described herein.

In some embodiments, the kit further comprises a container tube 112 as described herein. In some embodiments, the kit comprises 1, 2, 3, 4, 5, 6, 7, 8, 9, 10, 11, 12, 13, 14, 15, 16, 17, 18, 19, 20 or more container tubes 112 as described herein.

In some embodiments, the kit further comprises an effective amount of sample transport media. As will be appreciated by one of skill in the art, the sample transport media can be supplied in a lyophilized or dried form or a concentrated liquid form that can diluted or suspended in liquid prior to use with the swab 100. Preferred formulations include those that are non-toxic to the samples (e.g., cells, bacteria, viruses) and/or does not affect growth rate or viability. When the sample transport media is provided in a liquid solution, the liquid solution preferably is an aqueous solution, with a sterile aqueous solution being preferred. The sample transport media can be supplied in aliquots or in unit doses. In some embodiments of any of the aspects, transport media preserves the sample components (e.g., cellular, bacterial, or viral nucleic acids or polypeptides) nucleic acid between the time of sample collection and downstream applications.

In some embodiments of any of the aspects, the sample transport media comprises a viral transport media (VTM). The constituents of suitable viral transport media are designed to provide an isotonic solution containing protective protein, antibiotics to control microbial contamination, and one or more buffers to control the pH. Isotonicity, however, is not an absolute requirement; some highly successful transport media contain hypertonic solutions of sucrose. Liquid transport media are used primarily for transporting swabs 100 or materials released into the medium from a collection swab 100. Liquid media may be added to other specimens when inactivation of the viral agent is likely and when the resultant dilution is acceptable. A suitable VTM for use in collecting throat and nasal swabs from human patients is prepared as follows: (1) add 10 g veal infusion broth and 2 g bovine albumin fraction V to sterile distilled water (to 400 ml); (2) add 0.8 ml gentamicin sulfate solution (50 mg/ml) and 3.2 ml amphotericin B (250 µg/ml); and (3) sterilize by filtration. Additional non-limiting examples of viral transport media include COPAN Universal Transport Medium; Eagle Minimum Essential Medium (E-MEM); Transport medium 199; and PBS-Glycerol transport medium. see e.g., Johnson, Transport of Viral Specimens, CLINICAL MICROBIOLOGY REVIEWS, April 1990, p. 120-131; Collecting, preserving and shipping specimens for the diagnosis of avian influenza A(H5N1) virus infection, Guide for field operations, October 2006.

In some embodiments, the components described herein can be provided singularly or in any combination as a kit. Such a kit includes the components described herein, e.g., a swab 100, a container tube 112, and/or sample transport media, as described throughout the specification, or any combination thereof. Such kits can optionally include one or more agents that permit the detection of cellular, bacterial, or viral nucleic acids or polypeptides in the sample (e.g., test strips). In addition, the kit optionally comprises informational material.

In some embodiments, the compositions in the kit can be provided in a watertight or gas tight container which in some embodiments is substantially free of other components of the kit. For example, the swab 100 can be supplied in at least one container (e.g., the container tube 112), and the sample transport media can be supplied in a container having sufficient reagent for a predetermined number of samples, e.g., 1, 2, 3 or greater. It is preferred that the components described herein are substantially pure and/or sterile.

The informational material can be descriptive, instructional, marketing or other material that relates to the methods described herein. The informational material of the kits is not limited in its form. In one embodiment, the informational material can include information about production of any of the components (e.g., swabs 100, container tubes 112, sample transport media), concentration, date of expiration, batch or production site information, and so forth. In one embodiment, the informational material relates to methods for collecting samples using the components of the kit.

The kit will typically be provided with its various elements included in one package, e.g., a fiber-based, e.g., a cardboard, or polymeric, e.g., a Styrofoam box. The enclosure can be configured so as to maintain a temperature differential between the interior and the exterior, e.g., it can provide insulating properties to keep the reagents at a preselected temperature for a preselected time.

### Methods of Manufacture and Use

In some embodiments, the swab 100 is manufactured using injection molding, stamping, die cutting, thermal, ultrasonic welding, or 3D printing. In some embodiments, the swab 100 is injection molded. Accordingly, in one aspect described herein is method of manufacturing a swab 100 comprising: (a) injecting a mold with a liquid form of the swab material(s); and (b) removing the swab 100 from the mold once solidified. In some embodiments, the swab material is polypropylene. In some embodiments, the swab material is liquefied, e.g., at a temperature of about 150° C. In some embodiments, the step of removing the swab 100 from the mold comprises use of ejection pins, e.g., that contact at least one incomplete ring 115 of the sample collection head 102 as described herein. The method of manufacturing the swab 100 further comprises a first step of manufacturing the mold, e.g., according to the swab dimensions as described further herein.

In some embodiments, a swab 100 comprising at least two materials can be accomplished using injection molding (e.g., overmolding). Overmolding is a process wherein a single part is created using two or more different materials in combination. Typically, the first material, sometimes referred to as the substrate, is partially or fully covered by subsequent materials (e.g., overmold materials) during the manufacturing process.

In one aspect, described herein is a method of collecting a sample comprising contacting a sample with a swab 100 as described herein. The term “sample” as used herein denotes a sample taken or isolated from a biological organism, e.g., a blood or plasma sample from a subject. In some embodiments of any of the aspects, the present invention encompasses several examples of a biological sample. In some embodiments of any of the aspects, the biological sample is cells, or tissue, or peripheral blood, or bodily fluid. In some embodiments of any of the aspects, the biological sample comprises cells, mucus, and any microorganisms (e.g., bacteria, viruses, fungi). Exemplary biological samples include, but are not limited to, a biopsy, a tumor sample, biofluid sample; blood; serum; plasma; urine; semen; mucus; tissue biopsy; organ biopsy; synovial fluid; bile fluid; cerebrospinal fluid; mucosal secretion; effusion; sweat; saliva; and/or tissue sample etc. The term also includes a mixture of the above-mentioned samples. The term sample also includes untreated or pretreated (or pre-processed) biological samples. In some embodiments of any of the aspects, a sample can comprise cells from a subject. In some embodiments, the sample is selected from: nasopharyngeal, oropharyngeal, anterior nares, mid-turbinates, and buccal epithelial surface of a subject. In some embodiments, the sample is a nasopharyngeal epithelial surface of a subject. In some embodiments, the sample is an anterior nare epithelial surface of a subject.

In some embodiments, the subject is infected with or suspected to be infected with a respiratory infection. In some embodiments of any of the aspects, the respiratory infection is caused by a bacteria, virus, or fungus, e.g., which can replicate in the pulmonary and/or bronchial epithelia. Non-limiting examples of bacteria, virus, or fungi that can cause respiratory infections include: bacteria belonging to one of the Streptococcus, Haemophilus, Staphylococcus, or Moraxella genera (e.g., Streptococcus pneumoniae, Haemophilus influenzae, Staphylococcus aureus, or Moraxella catarrhalis), rhinoviruses (hRV), respiratory syncytial virus (RSV), adenoviruses (AdV), coronavirus (CoV), influenza viruses (IV), para-influenza viruses (PIV), human metapneumovirus (hMPV), or fungi belonging to the Aspergillus genus.

In some embodiments of any of the aspects, the respiratory infection is caused by a coronavirus. The scientific name for coronavirus is Orthocoronavirinae or Coronavirinae. Coronaviruses belong to the family of Coronaviridae, order Nidovirales, and realm Riboviria. They are divided into alphacoronaviruses and betacoronaviruses which infect mammals – and gammacoronaviruses and deltacoronaviruses which primarily infect birds. Non limiting examples of alphacoronaviruses include: Human coronavirus 229E, Human coronavirus NL63, Miniopterus bat coronavirus 1, Miniopterus bat coronavirus HKU8, Porcine epidemic diarrhea virus, Rhinolophus bat coronavirus HKU2, Scotophilus bat coronavirus 512, and Feline Infectious Peritonitis Virus (FIPV, also referred to as Feline Infectious Hepatitis Virus). Non limiting examples of betacoronaviruses include: Betacoronavirus 1 (e.g., Bovine Coronavirus, Human coronavirus OC43), Human coronavirus HKU1, Murine coronavirus (also known as Mouse hepatitis virus (MHV)), Pipistrellus bat coronavirus HKU5, Rousettus bat coronavirus HKU9, Severe acute respiratory syndrome-related coronavirus (e.g., SARS-CoV, SARS-CoV-2), Tylonycteris bat coronavirus HKU4, Middle East respiratory syndrome (MERS)-related coronavirus, and Hedgehog coronavirus 1 (EriCoV). Non limiting examples of gammacoronaviruses include: Beluga whale coronavirus SW1, and Infectious bronchitis virus. Non limiting examples of deltacoronaviruses include: Bulbul coronavirus HKU11, and Porcine coronavirus HKU15.

In some embodiments of any of the aspects, the coronavirus is selected from the group consisting of: severe acute respiratory syndrome-associated coronavirus (SARS-CoV); severe acute respiratory syndrome-associated coronavirus 2 (SARS-CoV-2); Middle East respiratory syndrome-related coronavirus (MERS-CoV); HCoV-NL63; and HCoV-HKu1. In some embodiments of any of the aspects, the coronavirus is severe acute respiratory syndrome coronavirus 2 (SARS-CoV-2), which causes coronavirus disease of 2019 (COVID19 or simply COVID). In some embodiments of any of the aspects, the coronavirus is severe acute respiratory syndrome coronavirus (SARS-CoV or SARS-CoV-1), which causes SARS. In some embodiments of any of the aspects, the coronavirus is Middle East respiratory syndrome-related coronavirus (MERS-CoV), which causes MERS.

In some embodiments after the contacting step, the swab 100 is separated into two pieces at the breakpoint 108. In some embodiments, after the contacting step, the swab 100 is separated into at least two pieces at the at least one breakpoint 108.

In some embodiments after the contacting step, the swab 100 is deposited into a container tube 112. In some embodiments, the container tube 112 contains sample transport media. In some embodiments after the contacting step, the swab 100 or at least a portion of the swab 100 (e.g., the soluble portion) is dissolved, e.g., with water or an aqueous solution if the swab material is water-soluble. Such a dissolving step can permit faster release of the sample from the swab 100 for downstream applications. In some embodiments, the downstream application comprises nucleic acid (e.g., RNA or DNA) extraction, protein extraction, nucleic acid (e.g., RNA or DNA) amplification (e.g., PCR or isothermal amplification methods). Non-limiting examples of isothermal amplification methods include: Recombinase Polymerase Amplification (RPA), Loop Mediated Isothermal Amplification (LAMP), Helicase-dependent isothermal DNA amplification (HDA), Rolling Circle Amplification (RCA), Nucleic acid sequence-based amplification (NASBA), strand displacement amplification (SDA), nicking enzyme amplification reaction (NEAR), and polymerase Spiral Reaction (PSR). In some embodiments, the downstream application is a diagnostic test, e.g., detection of nucleic acid or protein from at least one microbe of interest. In some embodiments, the downstream application is an automated diagnostic test.

In some embodiments, after a soluble swab 100 is dissolved in a buffer for a downstream application, the dissolved swab material (e.g., PVA) represents at most 22% (w/v) of the buffer. In some embodiments, the dissolved swab material (e.g., PVA) represents at most 1%, at most 2%, at most 3%, at most 4%, at most 5%, at most 6%, at most 7%, at most 8%, at most 9%, at most 10%, at most 11%, at most 12%, at most 13%, at most 14%, at most 15%, at most 16%, at most 17%, at most 18%, at most 19%, at most 20%, at most 21%, at most 22%, at most 23%, at most 24%, at most 25%, at most 26%, at most 27%, at most 28%, at most 29%, at most 30%, at most 31%, at most 32%, at most 33%, at most 34%, at most 35%, at most 36%, at most 37%, at most 38%, at most 39%, at most 40%, at most 41%, at most 42%, at most 43%, at most 44%, at most 45%, at most 46%, at most 47%, at most 48%, at most 49%, or at most 50% (w/v) of the buffer.

In some embodiments, the dissolved swab 100 does not inhibit or reduce a downstream application. In some embodiments, the dissolved swab 100 reduces a downstream application(s) by at most 1%, at most 2%, at most 3%, at most 4%, at most 5%, at most 6%, at most 7%, at most 8%, at most 9%, at most 10%, at most 11%, at most 12%, at most 13%, at most 14%, at most 15%, at most 16%, at most 17%, at most 18%, at most 19%, at most 20%, at most 21%, at most 22%, at most 23%, at most 24%, at most 25%, at most 26%, at most 27%, at most 28%, at most 29%, at most 30%, at most 31%, at most 32%, at most 33%, at most 34%, at most 35%, at most 36%, at most 37%, at most 38%, at most 39%, at most 40%, at most 41%, at most 42%, at most 43%, at most 44%, at most 45%, at most 46%, at most 47%, at most 48%, at most 49%, or at most 50% compared to a downstream application without the dissolved swab material.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of respiratory infections. A subject can be male or female.

A subject can be one who has been previously diagnosed with or identified as suffering from or having a respiratory infection or one or more complications related to such a respiratory infection, and optionally, have already undergone treatment for a respiratory infection or the one or more complications related to a respiratory infection. Alternatively, a subject can also be one who has not been previously diagnosed as having a respiratory infection or one or more complications related to a respiratory infection. For example, a subject can be one who exhibits one or more risk factors for a respiratory infection or one or more complications related to a respiratory infection or a subject who does not exhibit risk factors.

As used herein, “contacting” refers to any suitable means for delivering, or exposing, an agent to at least one cell. Exemplary delivery methods include, but are not limited to, direct delivery to cell culture medium, transfection, transduction, perfusion, injection, or other delivery method known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in cell biology, immunology, and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway’s Immunobiology, Kenneth Murphy, Allan Mowat, Casey Weaver (eds.), W. W. Norton & Company, 2016 (ISBN 0815345054, 978-0815345053); Lewin’s Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A swab comprising a sample collection head, the head comprising a plurality of spaced apart annular rings.
2. The swab of paragraph 1, wherein the plurality of rings are spaced 0.5 mm-2.0 mm.
3. The swab of paragraph 1 or 2, wherein the plurality of rings are spaced 0.75 mm.
4. The swab of any one of paragraphs 1-3, wherein the plurality of rings have a thickness of 0.1 mm-3.0 mm.
5. The swab of any one of paragraphs 1-4, wherein the plurality of rings have a thickness of 1.0 mm.
6. The swab of any one of paragraphs 1-5, wherein the plurality of rings have a diameter of 1.0 mm-4.0 mm.
7. The swab of any one of paragraphs 1-6, wherein the plurality of rings has a diameter of 2.5 mm.
8. The swab of any one of paragraphs 1-7, wherein the plurality of rings are tapered.
9. The swab of any one of paragraphs 1-8, wherein the plurality or rings have rounded edges.
10. The swab of any one of paragraphs 1-9, wherein the swab further comprises a handle and a neck, and wherein the head, handle, and neck comprise the same material.
11. The swab of any one of paragraphs 1-10, wherein at least one component of the swab is made from a different material from the remainder of the swab.
12. The swab of any one of paragraphs 1-11, wherein the swab is injection molded.
13. The swab of any one of paragraphs 1-12, wherein the material is a flexible polymer.
14. The swab of any one of paragraphs 1-13, wherein the material is polypropylene.
15. The swab of any one of paragraphs 1-14, wherein the material is biodegradable.
16. The swab of any one of paragraphs 1-15, wherein the material is water-soluble.
17. The swab of any one of paragraphs 1-16, wherein the material is polyvinyl alcohol (PVA).
18. The swab of any one of paragraphs 1-17, wherein the material is foam or a porous material.
19. The swab of any one of paragraphs 1-18, wherein the head does not comprise a fibrous coating.
20. The swab of any one of paragraphs 1-19, wherein the sample collection head comprises a first material, and the remainder of the swab comprises a second material.
21. The swab of any one of paragraphs 1-20, wherein the sample collection head comprises a water-soluble or biodegradable material and the remainder of the swab comprises a flexible polymer.
22. The swab of any one of paragraphs 1-21, wherein the sample collection head comprises PVA and the remainder of the swab comprises polypropylene.
23. The swab of any one of paragraphs 1-22, further comprising a neck, a plunger, and/or a flattened handle.
24. The swab of any one of paragraphs 1-23, wherein the neck tapers from a maximum diameter towards the handle to a minimum diameter towards the head.
25. The swab of any one of paragraphs 1-24, further comprising a breakpoint proximal to the head.
26. The swab of any one of paragraphs 1-25, wherein the head is stippled, roughened, or textured to increase surface area.
27. A swab for sample collection, wherein the swab is constructed from a water-soluble or biodegradable material.
28. The swab of paragraph 27, wherein the material is biodegradable and water-soluble.
29. The swab of any one of paragraphs 27-28, wherein the material is polyvinyl alcohol (PVA).
30. The swab of any one of paragraphs 27-29, wherein the material is foam or a porous material.
31. A swab for sample collection, wherein the swab is constructed from a flexible polymer and a water-soluble or biodegradable material.
32. The swab of paragraph 31, wherein the sample collection head comprises a water-soluble or biodegradable material and the remainder of the swab comprises a flexible polymer.
33. The swab of paragraph 31 or 32, wherein the sample collection head comprises PVA and the remainder of the swab comprises polypropylene.
34. The swab of any one of paragraphs 1-33, in combination with a container tube.
35. A kit comprising the swab of any one of paragraphs 1-33.
36. The kit of paragraph 28, further comprising a container tube and/or sample transport media.
37. A method of collecting a sample comprising contacting a sample with the swab of any one of paragraphs 1-34.
38. The method of paragraph 37, wherein the sample is selected from: nasopharyngeal, oropharyngeal, anterior nares, mid-turbinates, and buccal epithelial surface of a subject.
39. The method of paragraph 37 or 38, wherein the sample is a nasopharyngeal epithelial surface of a subject.
40. The method of any one of paragraphs 37-39, wherein the subject is infected with or suspected to be infected with a respiratory infection.
41. The method of any one of paragraphs 37-40, wherein after the contacting step, the swab is separated into two pieces at the breakpoint.
42. The method of any one of paragraphs 37-41, wherein after the contacting step, the swab is deposited into a container tube.
43. The method of any one of paragraphs 37-42, wherein the container tube contains sample transport media.
44. The method of any one of paragraphs 37-43, wherein after the contacting step, the swab or at least a portion of the swab is dissolved in a buffer.
45. The method of paragraph 44, wherein the dissolved swab represents at most 22% (w/v) of the buffer.
46. The method of paragraph 44 or 45, wherein the dissolved swab does not inhibit or reduce a downstream application.

### EXAMPLES

### Example 1: Nasopharyngeal Swab

### 1. Device Overview

Nasopharyngeal (NP) swabs are used to detect respiratory viruses, including but not limited to, SARS-CoV-2.

The NP swab described herein addresses the emergency shortage of conventional NP swabs experienced during the COVID-19 pandemic. However, the NP swab described herein can be used for any applicable in which a nasopharyngeal sample is needed.

The NP swab design exhibits at least the following attributes: (1) It is sufficiently rigid for collection of cells from the back of the throat. (2) It is sufficiently flexible for safety of use. (3) It collects adequate sample from the patient for subsequent tests (e.g., for viral infection). (4) It withstands the rigors of sterilization/disinfection without a) structural weakening, or b) chemically interfering with PCR testing. (5) It is compatible with standard PCR testing (e.g., for SARS-CoV-2). Furthermore, the swab can be mass-produced inexpensively to address swab shortages.

The swab described herein is an injection-molded, polypropylene NP swab (see e.g., FIG. 1). In some embodiments the NP swap is made-to specification, and is packaged in an off-the-shelf wrapper.

### 2. Use Case and Capability

This device was designed to address the imminent shortage of NP swabs for COVID-19 testing. It was designed as a permanent solution to the shortage. However, the NP swab described herein can be used for any applicable in which a nasopharyngeal sample is needed.

### 3. Validation Testing - Expert Evaluation, Collection Sufficiency, PCR Compatibility

The swab design was evaluated and approved by experts on the frontline of patient care, including infectious disease doctors, clinical pathologists, and respiratory therapists. The design was evaluated along the following parameters: expert evaluation, collection sufficiency, and PCR compatibility.

Mechanical efficacy and safety: Evaluators confirmed sufficient rigidity for sample collection from the back of the throat, in addition to sufficient flexibility for ensuring patient safety. Specific comment from evaluators: “Pass - appropriate body design, and tip head appropriately flexible and stiff.”

Collection sufficiency: Gram stains of the (inner) cheek made from the NP swab showed material broadly consistent with the control swab. Evaluators specifically reported that a) the NP swabs described herein were not too abrasive, and that b) they collected sufficient material.

PCR compatibility: PCR tested on an ABBOTT system.

The NP swab described herein meets the following Critical Design Inputs, as shown in Table 1.

TABLE 1

Nasopharyngeal Swab Critical Design Criteria

| | | |
|---|---|---|
| Critical Design Input | Pass/Fail | Notes |
| Sufficient mechanical rigidity for sample collection | Pass | Passing score assigned by expert evaluators |
| Sufficient mechanical flexibility for ensuring patient safety | Pass | Passing score assigned by expert evaluators |
| Facilitates collection of sufficient amount of sample for subsequent viral testing | Pass | Passing score assigned by expert evaluators; prototype NP swab result compared to those obtained using control NP swab |
| Compatible with PCR testing | Pass | PCR is tested using an ABBOTT system |
| Able to be sterilized/decontaminated | Pass | Standard polypropylene has been demonstrated to be sterilizable/decontaminatable via steam autoclave, gamma irradiation, and vaporized H₂O₂ |

### 4. Failure Mode Effect Analysis (FMEA)

Table 2 below shows a Failure Mode Effect Analysis (FMEA).

### 5. Manufacturability

The NP swab described herein is a single polypropylene unit that is manufactured via injection molding. The material comprises standard polypropylene. The requisite equipment comprises design molds. The manufacturing process comprises: Step 1: Make molds (e.g., 1-2 days); and Step 2: Injection-mold the NP swabs (e.g., ~10,000/day/mold cavity). Depending on the instrumentation and mold design, the production can be higher than 10,000/day/mold. As a non-limiting example, a mold can comprise multiple cavities such that each mold produces more than one swab at each molding. The estimated cost is approximately ~$0.50-$0.70 per swab.

TABLE 2

Failure Mode Effect Analysis (FMEA) (landscape view)

| | Potential Failure Mode | Potential Cause(s) | Detection Method | Potential Effects of Failure (Describe each time effect on device and patient/user.) | O (occurrence rating) | D (detection rating) | S (severity rating for patient) | C (critical characteristic) | Recommended Actions |
|---|---|---|---|---|---|---|---|---|---|
| Subcomponent | Method of failure | Why would this failure occur? | How would this failure be detected? | If this failure were to occur, what would happen? | How often this failure may occur, how easy it is to detect and the severity of this failure | | | | How does the design mitigate the failure or what should be tested does show this failure will not occur? Details of testing? |
| Design elements | Swab breakpoint does not allow for easy breakage | Swab design does not include a narrowing at the breakpoint | Inability to break prior to inserting used swab into test vial | A different swab would be used to test a patient (e.g., for COVID-19), or the patient would not be tested. | 1 in 100,000 | 1 | 5 | This failure mode is not associated with a critical characteristic. | The design includes a sharp narrowing at the recommended breakpoint. The prototype has been tested by several medical practitioners who confirm it can be consistently broken at this point. |
| Safety elements | Swab has rough edges that are abrasive against the patient’s nasal cavity and throat. | Swab design and/or injection molding procedure do not yield a smooth finish in the swab. | Patient expresses discomfort during nasopharyngeal swab procedure. | Swab design and/or fabrication method must be adjusted accordingly. | 1 in 50 | 1 | 6 | This failure mode is not associated with a critical characteristic. | The design was transitioned from a 3D-printed platform to an injection-molded platform in order to minimize sharp junctions. The prototype has been tested by several medical practitioners who suggest a smoother design, but ultimately confirm |

TABLE 2, CONT

Failure Mode Effect Analysis (FMEA) (landscape view)

| | | | | | | | | | |
|---|---|---|---|---|---|---|---|---|---|
| | Potential Failure Mode | Potential Cause(s) | Detection Method | Potential Effects of Failure (Describe each time effect on device and patient/user.) | O (occurrence rating) | D (detection rating) | S (severity rating for patient) | C (critical characteristic) | Recommended Actions |
| Safety elements | | | | | | | | | that the present design is ready for use in patients. |
| | Swab breaks inside patient during insertion procedure. | Manufacturing error passes undetected or swab is used incorrectly | Swab visibly breaks and/or patient expresses pain or discomfort | Detached swab piece must be retrieved from patient’s nasopharynx | 1 in 1,000,000 | 1 | 9 | Manufacturing accuracy | The prototype has been tested by several medical practitioners who confirm that the device’s rigidity is sufficient for safe testing in patients. |
| Human factors/end users | None | N/A | N/A | N/A | N/A | N/A | N/A | N/A | The prototype has been tested by several medical practitioners who confirm that use of the device is similar to use of traditional NP swabs. Only trained medical practitioners c use the NP swab. |

### Example 2: Innovative Concepts of the Swab

### Swab Head

The swab head permits collection of mucus, cells, and bacteria/viruses through the use of a high surface area textured head made of the same material as the handle and neck. Does not require flocking or other fibrous coating.

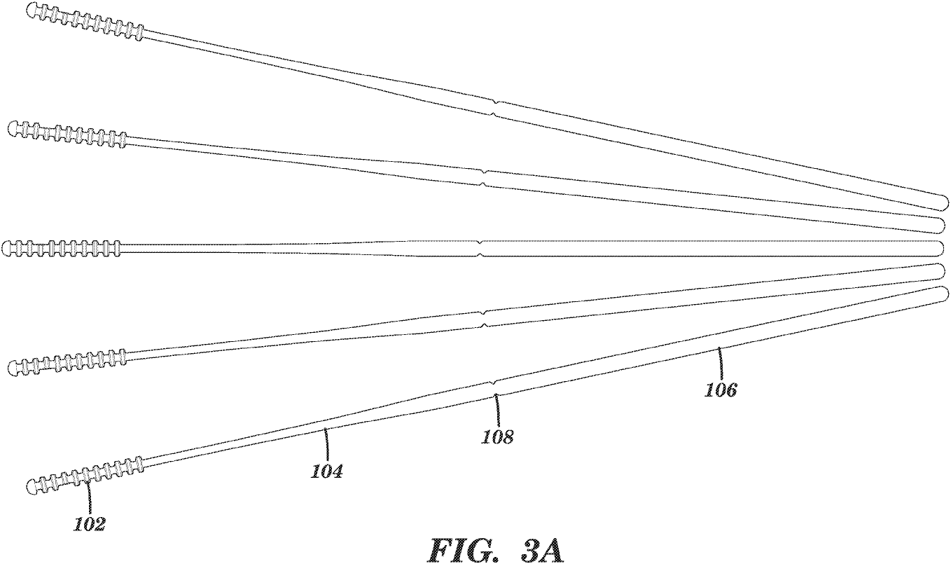

Testing has verified that the swab design is comfortable and is comparable to other swabs for its ability to capture and release RNA and does not inhibit downstream quantitative reverse transcription polymerase chain reaction (RT-qPCR), even without sample extraction (see e.g., FIG. 4). FIG. 4 is a bar graph 400 showing use of flocked and unflocked swabs to test for release of glyceraldehyde 3-phosphate dehydrogenase (GAPDH) from nasal samples from volunteers. The example NP swab described herein is #3. The other numbers are other swabs that have been approved for use. As shown, all swabs tested performed about the same in amount of GAPDH detected. The swabs performed comparably for capture and release whether by release by vortexing or spinning. Table 4 below shows the swabs tested in FIG. 4.

TABLE 4

Swabs tested in FIG. 4

| Swab # | Swab Description |
|---|---|
| 1 | Microbrush International™ |
| 2 | Plastcare USA™ (no bristles) |
| 3 | Swab as described herein |
| 4 | Puritan™ Sterile Foam Tipped Applicators™ |
| 5 | Puritan™ Sterile Polyester tipped applicators™ |
| 6 | Puritan™ hydraflock™ |
| 7 | Super brush™ 59-1187 |
| 8 | Super brush™ 59-4582 |
| 9 | BBL™ Culture swab™ |
| 10 | BCR™ Swab Lab Tips™ |
| 11 | Microbrush International™ |

The diameter of the head alternately increases and decreases along the longitudinal dimension of the head, e.g., from the proximal end of the head to the distal end of the head. The head thus forms a series of spaced annular rings, like a honey dipper for honey, that allow for capillary action of high-viscosity mucus to collect material. Rings also allow for gentle scraping of epithelium of the nasopharynx to abrade cells for analysis. Ring spacing of 0.5 mm-2 mm edge to edge (e.g., the gap between rings) is desirable to collect larger mucus and other material volumes while permitting holding it in place through capillary action. In one embodiment, the spacing is 0.75 mm gaps.

Ring thickness can be 0.1 mm-3.0 mm. Generally, relatively thinner rings permit greater head flexibility. In one embodiment, ring thickness is 1 mm. In one embodiment, the ring thickness is greater 0.3 mm. In addition, the rings can have rounded or eased edges. The rings can be tapered and, for example, increase or decrease in thickness towards their edges. In addition, the rings can have rounded or eased edges to facilitate insertion and withdrawal. The swab head can have one or more (e.g., a plurality of) rings. For example, the swab can have 2, 3, 4, 5, 6, 7, 8, 9, 10, 11, 12, 13, 14, 15, 16, 17, 18, 19, 20, 21, 22, 23, 24, 25, or more rings.

The rings can be circular, semicircular, truncated circles, or circles with one or more flat sides. In some embodiments, the rings can comprise a non-circular shape as described further herein. In addition, ring shape in a given swab head can be homogeneous or heterogeneous. In one embodiment, the head is tipped with a rounded bulb (e.g., a hemisphere or sphere), to facilitate insertion into the nasal cavity.

The head diameter (e.g., the diameter of the largest ring, or of the rounded bulb if the rounded bulb has a larger diameter than the largest ring) can be as small as 1.0 mm and can be up to 4 mm (e.g., max diameter to pass through nasal cavity). In one embodiment, head diameter is 1 mm, which was successfully tested. In one embodiment, head diameter is 2.5 mm. Without wishing to be bound by theory, it is thought that increased surface area of the head increases the collection amount and/or collection efficiency of the swab.

Lack of fibrous coating is better tolerated by patients since the swab is less likely to stick to the nasal passages when inserting swab.

A bead blast or other means of roughening the mold in the swab head region (or e.g., roughening each swab after molding) permits greater adhesion of fluids to the swab head. This roughening allowed the use of polypropylene, a very hydrophobic material that is more flexible than nylon, polystyrene, and other materials that are typically used for swabs.

A flexible polymer permits a conformal flex of the swab and exhibits the following characteristics: (1) Improved passage of swab through nasal cavity; (2) Reduced risk of injury to patient by deflecting with high loading force; and (3) Improved contact with nasopharynx over more rigid swab heads for sampling a larger surface area.

FHR P5M4R polypropylene copolymer with a tangent flexural modulus of 790 MPa was used. Moduli of less than 2000 MPa were tested, with 790 MPa, the softest material, performing best of 4 flexibilities tested. In some embodiments, the flexural moduli is in the 500-800 MPa range.

### Swab Neck

The neck comprises a gradual taper, from 2.5 mm at the handle to 1.2 mm at the start of the head, which was used to reduce stress concentration during use and increase ease of injection molding. The flexural modulus of the material is critical in providing adequate flex of the neck to reduce risk to the patient while providing adequate support for mucus and tissue collection. The modulus used (e.g., 790 MPa) was selected as the best performing one from 4 moduli tested by multiple clinicians.

### Additional Features and Geometries

Additional features were selected to be compatible with the existing viral swab testing pipeline and permit reach of a patient’s nasopharynx. The swab length is >140 mm. The swab breakpoint is at 70 mm-80 mm from swab head. The handle diameter is 2.5 mm. A large diameter is easier to hold. Diameter must be smaller than 4 mm to avoid damage to patient’s nasal cavity. The material is compatible with a range of sterilization modalities

Swab breakpoint combined with a less brittle polymer such as polypropylene can require a bend and twist action to remove head from handle

Swab head can be applied for other sampling sites: oropharyngeal, anterior nares, mid-turbinates, buccal. This involves adjusting geometry/material properties for appropriate stiffness needs.

Swab can be made from biodegradable material. Biodegradable materials include water soluble materials such as polyvinyl alcohol and derivative polymers. These soluble swabs can be used for dry swab transport for lower cost and greater stability. Soluble swabs can dissolve upon addition of reagents, permitting compatibility with automated fluid handling systems.

Swab heads without flocking or other fine fibers can permit faster release of pathogen and cellular material upon dry transfer by more easily dissociating mucus and other biopolymers from the dry sample

In one embodiment, the swab comprises an integrated plunger. Plunger-like feature provides stopping feature to avoid damage to nose by person performing swabbing (e.g., clinician or patient). In one embodiment, the integrated plunger is used as a stopper to use for dry transport.

In one embodiment, the swab is in combination with a container tube or cover. The tube can have flanges, grooves, or other geometric features to permit snapping or holding in place and/or sealing the swab and biological sample. Swab can contain a breakpoint. Swab can have integrated barcode or label. Swab could be made from water-soluble material and used in dry transport mode with cover.

### Example 3: Polyvinyl Alcohol (PVA) Swab

Described herein is a biodegradable, water-soluble swab 100 that can be constructed from PVA. Adding PVA to at least 22% by weight did not inhibit the qPCR reaction, the RPA reaction, or RNA extraction steps. PVA swabs 100 are thus compatible with multiple downstream applications.

A 22% PVA stock solution was prepared in viral transport media (VTM) or phosphate-buffered saline (PBS) buffer and heated at 95° C. for about 10 minutes, then vortexed briefly. The 22% stock was serially diluted to 16.5 %, 11%, and 5.5 %. PVA dilutions were mixed with 9:1 with RNA and RNase Inhibitor(RI) at concentrations of 10 \^4 mol/uL or 10 \^3 mol/uL. N-gene 1e5 mol/uL RNA was diluted in either PBS or VTM with Murine RNase Inhibitor. Final PVA Concentrations were 20%, 15%, 10%, or 5%. Final RNA Concentrations were 1000 mol/uL or 100 mol/uL.

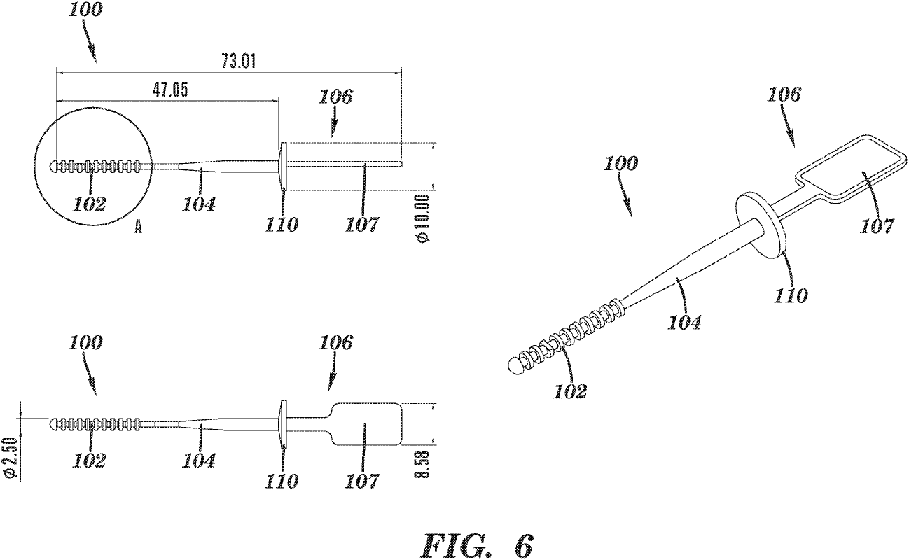

A first goal was to test whether PVA inhibits the standard RT-qPCR. What concentration of PVA results in the most amplification? PVA/RNA was input to a Luna™ RT-qPCR, using SARS-CoV-2 N gene primers JQ217 and JQ223, 8 uL of mastermix (MM), and 2 uL RNA/PVA input. FIG. 7 is a bar graph 700 showing PVA RT-qPCR compatibility. The x-axis indicates the final PVA percent concentration (w/v%) in the reaction. Bars within each PVA percentage group are in the same order left-right as the order of the legend left-right. As shown, the swabs 100 constructed from PVA did not inhibit the qPCR reaction.

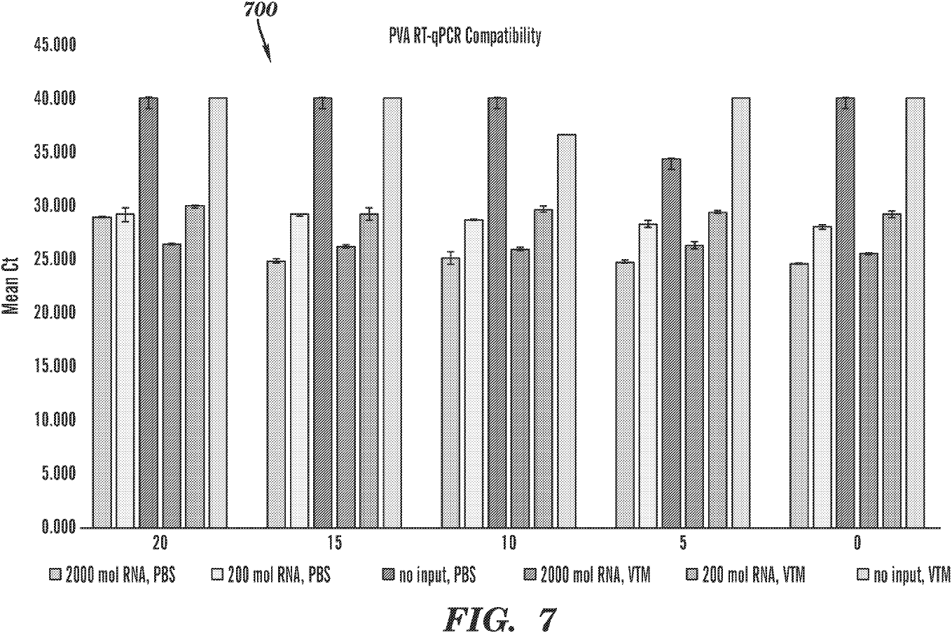

A second goal was to test whether PVA is compatible with the FIND (Fast Isothermal Nucleic acid Detection) assay (see e.g., Qian et al., An enhanced isothermal amplification assay for viral detection, May 2020, DOI: 10.1101/2020.05.28.118059, available on the world wide web at biorxiv.org/content/10.1101/2020.05.28.118059v1.full.pdf, the content of which is incorporated herein by reference in its entirety). PVA/RNA was input to N-gene recombinase polymerase amplification (RPA) and detected by quantitative polymerase chain reaction (qPCR). RPA used the JQ217 and JQ235 primers. The qPCR used the JQ289 and JQ223 primers. The N-gene RPA was run for 25 min at 42° C. (using the JQ217 and JQ235 primers; 8 uL MM; and 2 uL RNA/PVA Input). The RPA was diluted 1:200 for qPCR. The SybrGreen™ qPCR used the JQ289 and JQ223 primers, 10 uL MM, and 2 uL Input. All conditions with 0 molecules of input had a non-specific Tm. FIG. 8 is a line graph 800 showing PVA RPA-qPCR compatibility. As shown, swabs 100 constructed from PVA are compatible with the FIND assay.

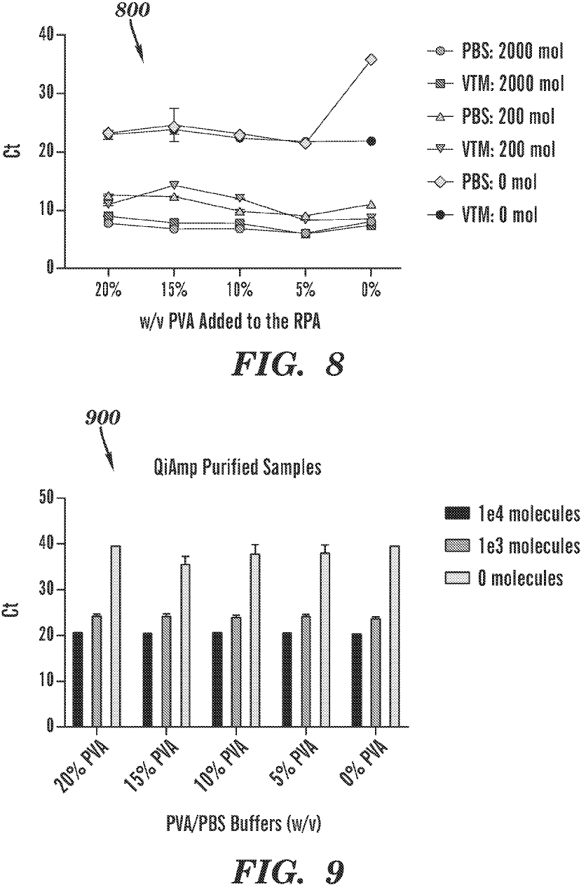

A third goal was to see if PVA inhibited RT-qPCR when utilizing a standard RNA purification protocol. PVA compatibility was first tested the using QIAmp™ Viral RNA Mini Kit. A 20% PVA buffer was prepared in 1x PBS, dissolved at 95° C. for about an hour. The PVA buffer was further diluted to 15%, 10%, and 5% (w/v PVA). N-gene RNA was diluted to 5e4 and 5e3 molecules/uL with Murine RNase Inhibitor (final concentration of 1 U/uL). N-gene RNA was mixed 1:10 with PVA buffer to reach a final volume of 140 uL. The samples were purified using QIAmp™ Viral RNA Mini Kit, following the manufacturer’s protocol. N-gene Luna™ RT-qPCR was run with JQ217 and JQ223 primers. FIG. 9 is a bar graph 900 showing PVA RT-qPCR compatibility using the QIAmp™ Viral RNA Mini Kit for RNA purification. As shown, swabs 100 constructed from PVA do not inhibit RT-qPCR when utilizing a standard RNA purification protocol.

1. A swab comprising a sample collection head, the head comprising a plurality of spaced apart annular rings.

2. The swab of claim 1, wherein the plurality of rings are spaced 0.5 mm - 2.0 mm.

3. The swab of claim 2, wherein the plurality of rings are spaced 0.75 mm.

4. The swab of claim 1, wherein the plurality of rings have a thickness of 0.1 mm - 3.0 mm.

5. The swab of claim 1, wherein the plurality of rings have a thickness of 1.0 mm.

6. The swab of claim 1, wherein the plurality of rings have a diameter of 1.0 mm - 4.0 mm.

7. The swab of claim 1, wherein the plurality of rings has a diameter of 2.5 mm.

8. The swab of claim 1, wherein the plurality of rings are tapered.

9. The swab of claim 1, wherein the plurality or rings have rounded edges.

10. The swab of claim 1, wherein the swab further comprises a handle and a neck, and wherein the head, handle, and neck comprise the same material.

11. The swab of claim 1, wherein at least one component of the swab is made from a different material from the remainder of the swab.

12. The swab of claim 1, wherein the swab is injection molded.

13. The swab of claim 1, wherein the material is a flexible polymer.

14. The swab of claim 1, wherein the material is polypropylene.

15. The swab of claim 1, wherein the material is biodegradable.

16. The swab of claim 1, wherein the material is water-soluble.

17. The swab of claim 1, wherein the material is polyvinyl alcohol (PV A).

18. The swab of claim 1, wherein the material is foam or a porous material.

19. The swab of claim 1, wherein the head does not comprise a fibrous coating.

20. The swab of claim 1, wherein the sample collection head comprises a first material, and the remainder of the swab comprises a second material.

21. The swab of claim 1, wherein the sample collection head comprises a water-soluble or biodegradable material and the remainder of the swab comprises a flexible polymer.

22. The swab of claim 1, wherein the sample collection head comprises PVA and the remainder of the swab comprises polypropylene.

23. The swab of claim 1, further comprising a neck, a plunger, and/or a flattened handle.

24. The swab of claim 1, wherein the neck tapers from a maximum diameter towards the handle to a minimum diameter towards the head.

25. The swab of claim 1, further comprising a breakpoint proximal to the head.

26. The swab of claim 1, wherein the head is stippled, roughened, or textured to increase surface area.

27. A swab for sample collection, wherein the swab is constructed from a water-soluble or biodegradable material.

28-30. (canceled)

31. A swab for sample collection, wherein the swab is constructed from a flexible polymer and a water-soluble or biodegradable material.

32-34. (canceled)

35. A kit comprising the swab of claim 1. .

36. (canceled)

37. A method of collecting a sample comprising contacting a sample with the swab of claim 1.

38-46. (canceled)
